SEQUENCE LISTING (214 sequences)

SEQ ID NO: 1 - DNA, 24 bp, synthetic construct
  source 1..24: mol_type = other DNA
caccgagtatgaggcatagactgc

SEQ ID NO: 2 - DNA, 24 bp, synthetic construct
  source 1..24: mol_type = other DNA
aaacgcagtctatgcctcatactc

SEQ ID NO: 3 - DNA, 24 bp, synthetic construct
  source 1..24: mol_type = other DNA
caccgagcaaagagaatagactgt

SEQ ID NO: 4 - DNA, 24 bp, synthetic construct
  source 1..24: mol_type = other DNA
aaacacagtctattctctttgctc

SEQ ID NO: 5 - DNA, 24 bp, synthetic construct
  source 1..24: mol_type = other DNA
caccgatgagataatgatgagtca

SEQ ID NO: 6 - DNA, 24 bp, synthetic construct
  source 1..24: mol_type = other DNA
aaactgactcatcattatctcatc

SEQ ID NO: 7 - DNA, 24 bp, synthetic construct
  source 1..24: mol_type = other DNA
caccgtaaacaaagcatagactga

SEQ ID NO: 8 - DNA, 24 bp, synthetic construct
  source 1..24: mol_type = other DNA
aaactcagtctatgctttgtttac

SEQ ID NO: 9 - DNA, 24 bp, synthetic construct
  source 1..24: mol_type = other DNA
caccgaacataaagaatagaatga

SEQ ID NO: 10 - DNA, 24 bp, synthetic construct
  source 1..24: mol_type = other DNA
aaactcattctattctttatgttc

SEQ ID NO: 11 - DNA, 24 bp, synthetic construct
  source 1..24: mol_type = other DNA
caccgaagatagagaatagactgc

SEQ ID NO: 12 - DNA, 24 bp, synthetic construct
  source 1..24: mol_type = other DNA
aaacgcagtctattctctatcttc

SEQ ID NO: 13 - DNA, 24 bp, synthetic construct
  source 1..24: mol_type = other DNA
caccgggaataaatcatagaatcc

SEQ ID NO: 14 - DNA, 24 bp, synthetic construct
  source 1..24: mol_type = other DNA
aaacggattctatgatttattccc

SEQ ID NO: 15 - DNA, 24 bp, synthetic construct
  source 1..24: mol_type = other DNA
caccgacaaagaggaagagagacg

SEQ ID NO: 16 - DNA, 24 bp, synthetic construct
  source 1..24: mol_type = other DNA
aaaccgtctctcttcctctttgtc

SEQ ID NO: 17 - DNA, 24 bp, synthetic construct
  source 1..24: mol_type = other DNA
caccgacaaaccagaagccgctcc

SEQ ID NO: 18 - DNA, 24 bp, synthetic construct
  source 1..24: mol_type = other DNA
aaacggagcggcttctggtttgtc

SEQ ID NO: 19 - DNA, 24 bp, synthetic construct
  source 1..24: mol_type = other DNA
caccggtgacaagtgtgatcactt

SEQ ID NO: 20 - DNA, 24 bp, synthetic construct
  source 1..24: mol_type = other DNA
aaacaagtgatcacacttgtcacc

SEQ ID NO: 21 - DNA, 24 bp, synthetic construct
  source 1..24: mol_type = other DNA
caccggaatcccttctgcagcacc

SEQ ID NO: 22 - DNA, 24 bp, synthetic construct
  source 1..24: mol_type = other DNA
aaacggtgctgcagaagggattcc

SEQ ID NO: 23 - DNA, 25 bp, synthetic construct
  source 1..25: mol_type = other DNA
caccgcttgaccaatagccttgaca

SEQ ID NO: 24 - DNA, 25 bp, synthetic construct
  source 1..25: mol_type = other DNA
aaactgtcaaggctattggtcaagc

SEQ ID NO: 25 - DNA, 25 bp, synthetic construct
  source 1..25: mol_type = other DNA
caccgatatttgcattgagatagtg

SEQ ID NO: 26 - DNA, 25 bp, synthetic construct
  source 1..25: mol_type = other DNA
aaaccactatctcaatgcaaatatc

SEQ ID NO: 27 - DNA, 24 bp, synthetic construct
  source 1..24: mol_type = other DNA
caccgtggggaaggggcccccaag

SEQ ID NO: 28 - DNA, 24 bp, synthetic construct
  source 1..24: mol_type = other DNA
aaaccttgggggccccttccccac

SEQ ID NO: 29 - DNA, 24 bp, synthetic construct
  source 1..24: mol_type = other DNA
caccgaacacaaagcatagactgc

SEQ ID NO: 30 - DNA, 24 bp, synthetic construct
  source 1..24: mol_type = other DNA
aaacgcagtctatgctttgtgttc

SEQ ID NO: 31 - DNA, 24 bp, synthetic construct
  source 1..24: mol_type = other DNA
caccggcccagactgagcacgtga

SEQ ID NO: 32 - DNA, 24 bp, synthetic construct
  source 1..24: mol_type = other DNA
aaactcacgtgctcagtctgggcc

SEQ ID NO: 33 - DNA, 24 bp, synthetic construct
  source 1..24: mol_type = other DNA
caccggcactgcggctggaggtcc

SEQ ID NO: 34 - DNA, 24 bp, synthetic construct
  source 1..24: mol_type = other DNA
aaacggacctccagccgcagtgcc

SEQ ID NO: 35 - DNA, 24 bp, synthetic construct
  source 1..24: mol_type = other DNA
caccggggacataaaagtaattgg

SEQ ID NO: 36 - DNA, 24 bp, synthetic construct
  source 1..24: mol_type = other DNA
aaacccaattacttttatgtcccc

SEQ ID NO: 37 - DNA, 24 bp, synthetic construct
  source 1..24: mol_type = other DNA
caccgaagtattcattatagtcaa

SEQ ID NO: 38 - DNA, 24 bp, synthetic construct
  source 1..24: mol_type = other DNA
aaacttgactataatgaatacttc

SEQ ID NO: 39 - DNA, 24 bp, synthetic construct
  source 1..24: mol_type = other DNA
caccggtgagtgagtgtgtgcgtg

SEQ ID NO: 40 - DNA, 24 bp, synthetic construct
  source 1..24: mol_type = other DNA
aaaccacgcacacactcactcacc

SEQ ID NO: 41 - DNA, 24 bp, synthetic construct
  source 1..24: mol_type = other DNA
caccgaccctcagccgtgctgctc

SEQ ID NO: 42 - DNA, 24 bp, synthetic construct
  source 1..24: mol_type = other DNA
aaacgagcagcacggctgagggtc

SEQ ID NO: 43 - DNA, 24 bp, synthetic construct
  source 1..24: mol_type = other DNA
caccgctctcagcctggagaccac

SEQ ID NO: 44 - DNA, 24 bp, synthetic construct
  source 1..24: mol_type = other DNA
aaacgtggtctccaggctgagagc

SEQ ID NO: 45 - DNA, 24 bp, synthetic construct
  source 1..24: mol_type = other DNA
caccggggctcaacatcggaagag

SEQ ID NO: 46 - DNA, 24 bp, synthetic construct
  source 1..24: mol_type = other DNA
aaacctcttccgatgttgagcccc

SEQ ID NO: 47 - DNA, 24 bp, synthetic construct
  source 1..24: mol_type = other DNA
caccgctggctcaggttcaggaga

SEQ ID NO: 48 - DNA, 24 bp, synthetic construct
  source 1..24: mol_type = other DNA
aaactctcctgaacctgagccagc

SEQ ID NO: 49 - DNA, 24 bp, synthetic construct
  source 1..24: mol_type = other DNA
caccgagtccgagcagaagaagaa

SEQ ID NO: 50 - DNA, 24 bp, synthetic construct
  source 1..24: mol_type = other DNA
aaacttcttcttctgctcggactc

SEQ ID NO: 51 - DNA, 24 bp, synthetic construct
  source 1..24: mol_type = other DNA
caccgcagggatccaagcacacaa

SEQ ID NO: 52 - DNA, 24 bp, synthetic construct
  source 1..24: mol_type = other DNA
aaacttgtgtgcttggatccctgc

SEQ ID NO: 53 - DNA, 24 bp, synthetic construct
  source 1..24: mol_type = other DNA
caccgctctcaggccctgtccgca

SEQ ID NO: 54 - DNA, 24 bp, synthetic construct
  source 1..24: mol_type = other DNA
aaactgcggacagggcctgagagc

SEQ ID NO: 55 - DNA, 24 bp, synthetic construct
  source 1..24: mol_type = other DNA
caccgagcaaataccagagataag

SEQ ID NO: 56 - DNA, 24 bp, synthetic construct
  source 1..24: mol_type = other DNA
aaaccttatctctggtatttgctc

SEQ ID NO: 57 - DNA, 24 bp, synthetic construct
  source 1..24: mol_type = other DNA
caccgagaacccaaatctccagga

SEQ ID NO: 58 - DNA, 24 bp, synthetic construct
  source 1..24: mol_type = other DNA
aaactcctggagatttgggttctc

SEQ ID NO: 59 - DNA, 24 bp, synthetic construct
  source 1..24: mol_type = other DNA
caccgtacctaatggacttcaggg

SEQ ID NO: 60 - DNA, 24 bp, synthetic construct
  source 1..24: mol_type = other DNA
aaacccctgaagtccattaggtac

SEQ ID NO: 61 - DNA, 24 bp, synthetic construct
  source 1..24: mol_type = other DNA
caccgccttcatagaattgaagag

SEQ ID NO: 62 - DNA, 24 bp, synthetic construct
  source 1..24: mol_type = other DNA
aaacctcttcaattctatgaaggc

SEQ ID NO: 63 - DNA, 93 bp, synthetic construct
  source 1..93: mol_type = other DNA
aaaaaaagcaccgactcggtgccactttttcaagttgataacggactagccttatttaaacttgctatgctgtttccagcatagctcttaaac

SEQ ID NO: 64 - DNA, 64 bp, synthetic construct
  source 1..64: mol_type = other DNA
gaaattaatacgactcactatagagtatgaggcatagactgcgtttaagagctatgctggaaac

SEQ ID NO: 65 - DNA, 64 bp, synthetic construct
  source 1..64: mol_type = other DNA
gaaattaatacgactcactatagagcaaagagaatagactgtgtttaagagctatgctggaaac

SEQ ID NO: 66 - DNA, 64 bp, synthetic construct
  source 1..64: mol_type = other DNA
gaaattaatacgactcactatagatgagataatgatgagtcagtttaagagctatgctggaaac

SEQ ID NO: 67 - DNA, 64 bp, synthetic construct
  source 1..64: mol_type = other DNA
gaaattaatacgactcactatagtaaacaaagcatagactgagtttaagagctatgctggaaac

SEQ ID NO: 68 - DNA, 64 bp, synthetic construct
  source 1..64: mol_type = other DNA
gaaattaatacgactcactatagaacataaagaatagaatgagtttaagagctatgctggaaac

SEQ ID NO: 69 - DNA, 64 bp, synthetic construct
  source 1..64: mol_type = other DNA
gaaattaatacgactcactatagaagatagagaatagactgcgtttaagagctatgctggaaac

SEQ ID NO: 70 - DNA, 64 bp, synthetic construct
  source 1..64: mol_type = other DNA
gaaattaatacgactcactatagggaataaatcatagaatccgtttaagagctatgctggaaac

SEQ ID NO: 71 - DNA, 64 bp, synthetic construct
  source 1..64: mol_type = other DNA
gaaattaatacgactcactatagacaaagaggaagagagacggtttaagagctatgctggaaac

SEQ ID NO: 72 - DNA, 64 bp, synthetic construct
  source 1..64: mol_type = other DNA
gaaattaatacgactcactatagacaaaccagaagccgctccgtttaagagctatgctggaaac

SEQ ID NO: 73 - DNA, 64 bp, synthetic construct
  source 1..64: mol_type = other DNA
gaaattaatacgactcactataggtgacaagtgtgatcacttgtttaagagctatgctggaaac

SEQ ID NO: 74 - DNA, 64 bp, synthetic construct
  source 1..64: mol_type = other DNA
gaaattaatacgactcactataggaatcccttctgcagcaccgtttaagagctatgctggaaac

SEQ ID NO: 75 - DNA, 65 bp, synthetic construct
  source 1..65: mol_type = other DNA
gaaattaatacgactcactatagcttgaccaatagccttgacagtttaagagctatgctggaaac

SEQ ID NO: 76 - DNA, 65 bp, synthetic construct
  source 1..65: mol_type = other DNA
gaaattaatacgactcactatagatatttgcattgagatagtggtttaagagctatgctggaaac

SEQ ID NO: 77 - DNA, 64 bp, synthetic construct
  source 1..64: mol_type = other DNA
gaaattaatacgactcactatagtggggaaggggcccccaaggtttaagagctatgctggaaac

SEQ ID NO: 78 - DNA, 64 bp, synthetic construct
  source 1..64: mol_type = other DNA
gaaattaatacgactcactatagaacacaaagcatagactgcgtttaagagctatgctggaaac

SEQ ID NO: 79 - DNA, 64 bp, synthetic construct
  source 1..64: mol_type = other DNA
gaaattaatacgactcactataggcccagactgagcacgtgagtttaagagctatgctggaaac

SEQ ID NO: 80 - DNA, 64 bp, synthetic construct
  source 1..64: mol_type = other DNA
gaaattaatacgactcactataggcactgcggctggaggtccgtttaagagctatgctggaaac

SEQ ID NO: 81 - DNA, 64 bp, synthetic construct
  source 1..64: mol_type = other DNA
gaaattaatacgactcactataggggacataaaagtaattgggtttaagagctatgctggaaac

SEQ ID NO: 82 - DNA, 64 bp, synthetic construct
  source 1..64: mol_type = other DNA
gaaattaatacgactcactatagaagtattcattatagtcaagtttaagagctatgctggaaac

SEQ ID NO: 83 - DNA, 64 bp, synthetic construct
  source 1..64: mol_type = other DNA
gaaattaatacgactcactataggtgagtgagtgtgtgcgtggtttaagagctatgctggaaac

SEQ ID NO: 84 - DNA, 64 bp, synthetic construct
  source 1..64: mol_type = other DNA
gaaattaatacgactcactatagaccctcagccgtgctgctcgtttaagagctatgctggaaac

SEQ ID NO: 85 - DNA, 64 bp, synthetic construct
  source 1..64: mol_type = other DNA
gaaattaatacgactcactatagctctcagcctggagaccacgtttaagagctatgctggaaac

SEQ ID NO: 86 - DNA, 64 bp, synthetic construct
  source 1..64: mol_type = other DNA
gaaattaatacgactcactataggggctcaacatcggaagaggtttaagagctatgctggaaac

SEQ ID NO: 87 - DNA, 64 bp, synthetic construct
  source 1..64: mol_type = other DNA
gaaattaatacgactcactatagctggctcaggttcaggagagtttaagagctatgctggaaac

SEQ ID NO: 88 - DNA, 64 bp, synthetic construct
  source 1..64: mol_type = other DNA
gaaattaatacgactcactatagagtccgagcagaagaagaagtttaagagctatgctggaaac

SEQ ID NO: 89 - DNA, 64 bp, synthetic construct
  source 1..64: mol_type = other DNA
gaaattaatacgactcactatagcagggatccaagcacacaagtttaagagctatgctggaaac

SEQ ID NO: 90 - DNA, 64 bp, synthetic construct
  source 1..64: mol_type = other DNA
gaaattaatacgactcactatagctctcaggccctgtccgcagtttaagagctatgctggaaac

SEQ ID NO: 91 - DNA, 64 bp, synthetic construct
  source 1..64: mol_type = other DNA
gaaattaatacgactcactatagagcaaataccagagataaggtttaagagctatgctggaaac

SEQ ID NO: 92 - DNA, 64 bp, synthetic construct
  source 1..64: mol_type = other DNA
gaaattaatacgactcactatagagaacccaaatctccaggagtttaagagctatgctggaaac

SEQ ID NO: 93 - DNA, 64 bp, synthetic construct
  source 1..64: mol_type = other DNA
gaaattaatacgactcactatagtacctaatggacttcaggggtttaagagctatgctggaaac

SEQ ID NO: 94 - DNA, 64 bp, synthetic construct
  source 1..64: mol_type = other DNA
gaaattaatacgactcactatagccttcatagaattgaagaggtttaagagctatgctggaaac

SEQ ID NO: 95 - DNA, 65 bp, synthetic construct
  source 1..65: mol_type = other DNA
gaaattaatacgactcactatagacatcagaggagactgccaggtttaagagctatgctggaaac

SEQ ID NO: 96 - DNA, 64 bp, synthetic construct
  source 1..64: mol_type = other DNA
gaaattaatacgactcactatagcatcagaggagactgccaggtttaagagctatgctggaaac

SEQ ID NO: 97 - DNA, 54 bp, synthetic construct
  source 1..54: mol_type = other DNA
acactctttccctacacgacgctcttccgatctgggaacctcaggtgaaaagtc

SEQ ID NO: 98 - DNA, 54 bp, synthetic construct
  source 1..54: mol_type = other DNA
gtgactggagttcagacgtgtgctcttccgatctcctaggcaacaagagcaaaa

SEQ ID NO: 99 - DNA, 53 bp, synthetic construct
  source 1..53: mol_type = other DNA
acactctttccctacacgacgctcttccgatcttctcgatctcctgacctcgt

SEQ ID NO: 100 - DNA, 59 bp, synthetic construct
  source 1..59: mol_type = other DNA
gtgactggagttcagacgtgtgctcttccgatcttgtcaggtaatgtgctaaacagaga

SEQ ID NO: 101 - DNA, 53 bp, synthetic construct
  source 1..53: mol_type = other DNA
acactctttccctacacgacgctcttccgatctaagtcaagcctgagcttcca

SEQ ID NO: 102 - DNA, 54 bp, synthetic construct
  source 1..54: mol_type = other DNA
gtgactggagttcagacgtgtgctcttccgatctcccaaagtgctgggatttta

SEQ ID NO: 103 - DNA, 53 bp, synthetic construct
  source 1..53: mol_type = other DNA
acactctttccctacacgacgctcttccgatctgccgtgggagacaattcata

SEQ ID NO: 104 - DNA, 53 bp, synthetic construct
  source 1..53: mol_type = other DNA
gtgactggagttcagacgtgtgctcttccgatctggttctgtttgtggccaag

SEQ ID NO: 105 - DNA, 58 bp, synthetic construct
  source 1..58: mol_type = other DNA
acactctttccctacacgacgctcttccgatctaaagtcactctgctgtgctattaga

SEQ ID NO: 106 - DNA, 54 bp, synthetic construct
  source 1..54: mol_type = other DNA
gtgactggagttcagacgtgtgctcttccgatcttgagcaaggcacagaaagac

SEQ ID NO: 107 - DNA, 57 bp, synthetic construct
  source 1..57: mol_type = other DNA
acactctttccctacacgacgctcttccgatcttcaaacactgggtcatacttcttc

SEQ ID NO: 108 - DNA, 54 bp, synthetic construct
  source 1..54: mol_type = other DNA
gtgactggagttcagacgtgtgctcttccgatctccatctgggcacagtgttac

SEQ ID NO: 109 - DNA, 53 bp, synthetic construct
  source 1..53: mol_type = other DNA
acactctttccctacacgacgctcttccgatctccacctggaatgagttttcg

SEQ ID NO: 110 - DNA, 54 bp, synthetic construct
  source 1..54: mol_type = other DNA
gtgactggagttcagacgtgtgctcttccgatctatgccagataccagcaatcc

SEQ ID NO: 111 - DNA, 53 bp, synthetic construct
  source 1..53: mol_type = other DNA
acactctttccctacacgacgctcttccgatctcaagcctgattccaaggaga

SEQ ID NO: 112 - DNA, 54 bp, synthetic construct
  source 1..54: mol_type = other DNA
gtgactggagttcagacgtgtgctcttccgatctaaatgagcctctggtggaga

SEQ ID NO: 113 - DNA, 57 bp, synthetic construct
  source 1..57: mol_type = other DNA
acactctttccctacacgacgctcttccgatctttcttgtagccctctttttattgg

SEQ ID NO: 114 - DNA, 56 bp, synthetic construct
  source 1..56: mol_type = other DNA
gtgactggagttcagacgtgtgctcttccgatctcctatcacaatcacaactgcaa

SEQ ID NO: 115 - DNA, 53 bp, synthetic construct
  source 1..53: mol_type = other DNA
acactctttccctacacgacgctcttccgatctcctgacaatcgataggtacc

SEQ ID NO: 116 - DNA, 54 bp, synthetic construct
  source 1..54: mol_type = other DNA
gtgactggagttcagacgtgtgctcttccgatctagccccaagatgactatctt

SEQ ID NO: 117 - DNA, 53 bp, synthetic construct
  source 1..53: mol_type = other DNA
acactctttccctacacgacgctcttccgatctagcattgcagagaggcgtat

SEQ ID NO: 118 - DNA, 53 bp, synthetic construct
  source 1..53: mol_type = other DNA
gtgactggagttcagacgtgtgctcttccgatctatggatgtggcgcaggtag

SEQ ID NO: 119 - DNA, 56 bp, synthetic construct
  source 1..56: mol_type = other DNA
acactctttccctacacgacgctcttccgatctccttttattcttcatccctagcc

SEQ ID NO: 120 - DNA, 55 bp, synthetic construct
  source 1..55: mol_type = other DNA
gtgactggagttcagacgtgtgctcttccgatctggaatgactgaatcggaacaa

SEQ ID NO: 121 - DNA, 54 bp, synthetic construct
  source 1..54: mol_type = other DNA
acactctttccctacacgacgctcttccgatctggacgtctgcccaatatgtaa

SEQ ID NO: 122 - DNA, 54 bp, synthetic construct
  source 1..54: mol_type = other DNA
gtgactggagttcagacgtgtgctcttccgatctaagggggaaaaattgtccag

SEQ ID NO: 123 - DNA, 53 bp, synthetic construct
  source 1..53: mol_type = other DNA
acactctttccctacacgacgctcttccgatctaaacgcccatgcaattagtc

SEQ ID NO: 124 - DNA, 53 bp, synthetic construct
  source 1..53: mol_type = other DNA
gtgactggagttcagacgtgtgctcttccgatctccagccaaacttgtcaacc

SEQ ID NO: 125 - DNA, 53 bp, synthetic construct
  source 1..53: mol_type = other DNA
acactctttccctacacgacgctcttccgatctctcccttcaagatggctgac

SEQ ID NO: 126 - DNA, 54 bp, synthetic construct
  source 1..54: mol_type = other DNA
gtgactggagttcagacgtgtgctcttccgatctaacggagacacacacacagg

SEQ ID NO: 127 - DNA, 59 bp, synthetic construct
  source 1..59: mol_type = other DNA
acactctttccctacacgacgctcttccgatctttgaagtttgtgtgtgtacataagga

SEQ ID NO: 128 - DNA, 57 bp, synthetic construct
  source 1..57: mol_type = other DNA
gtgactggagttcagacgtgtgctcttccgatcttgattgattgaaagcacactgtt

SEQ ID NO: 129 - DNA, 54 bp, synthetic construct
  source 1..54: mol_type = other DNA
acactctttccctacacgacgctcttccgatctgagaggcacatttgccagtat

SEQ ID NO: 130 - DNA, 55 bp, synthetic construct
  source 1..55: mol_type = other DNA
gtgactggagttcagacgtgtgctcttccgatcttatgaggtgctggaaggagaa

SEQ ID NO: 131 - DNA, 52 bp, synthetic construct
  source 1..52: mol_type = other DNA
acactctttccctacacgacgctcttccgatctacagggaagctgggtgaat

SEQ ID NO: 132 - DNA, 54 bp, synthetic construct
  source 1..54: mol_type = other DNA
gtgactggagttcagacgtgtgctcttccgatctcacacgcacacactcactca

SEQ ID NO: 133 - DNA, 60 bp, synthetic construct
  source 1..60: mol_type = other DNA
acactctttccctacacgacgctcttccgatctggaattctgcatcatgtgggcgtgtcc

SEQ ID NO: 134 - DNA, 61 bp, synthetic construct
  source 1..61: mol_type = other DNA
gtgactggagttcagacgtgtgctcttccgatctggatcagccaggaacgaagaccactgg

SEQ ID NO: 135 - DNA, 60 bp, synthetic construct
  source 1..60: mol_type = other DNA
acactctttccctacacgacgctcttccgatctaagacaatcctaggaagcagggtcagc

SEQ ID NO: 136 - DNA, 52 bp, synthetic construct
  source 1..52: mol_type = other DNA
gtgactggagttcagacgtgtgctcttccgatctgggccaacatcgccgccg

SEQ ID NO: 137 - DNA, 58 bp, synthetic construct
  source 1..58: mol_type = other DNA
acactctttccctacacgacgctcttccgatctgccaggcagatagaccagactgagc

SEQ ID NO: 138 - DNA, 59 bp, synthetic construct
  source 1..59: mol_type = other DNA
gtgactggagttcagacgtgtgctcttccgatctgcctccttaccattccccttcgacc

SEQ ID NO: 139 - DNA, 53 bp, synthetic construct
  source 1..53: mol_type = other DNA
acactctttccctacacgacgctcttccgatctttcttcccacctggaatgtc

SEQ ID NO: 140 - DNA, 55 bp, synthetic construct
  source 1..55: mol_type = other DNA
gtgactggagttcagacgtgtgctcttccgatctgcactgactggacacaagtga

SEQ ID NO: 141 - DNA, 55 bp, synthetic construct
  source 1..55: mol_type = other DNA
acactctttccctacacgacgctcttccgatctggtcactccaggattccaatag

SEQ ID NO: 142 - DNA, 54 bp, synthetic construct
  source 1..54: mol_type = other DNA
gtgactggagttcagacgtgtgctcttccgatctccaaggttcacagcctgaaa

SEQ ID NO: 143 - DNA, 53 bp, synthetic construct
  source 1..53: mol_type = other DNA
acactctttccctacacgacgctcttccgatctcctcctttgtctctgcctgt

SEQ ID NO: 144 - DNA, 59 bp, synthetic construct
  source 1..59: mol_type = other DNA
gtgactggagttcagacgtgtgctcttccgatctggatctacatcacgtaacctgtctt

SEQ ID NO: 145 - DNA, 53 bp, synthetic construct
  source 1..53: mol_type = other DNA
acactctttccctacacgacgctcttccgatctccgatgaggagacactccaa

SEQ ID NO: 146 - DNA, 53 bp, synthetic construct
  source 1..53: mol_type = other DNA
gtgactggagttcagacgtgtgctcttccgatctccacaggctgctgagaaac

SEQ ID NO: 147 - DNA, 57 bp, synthetic construct
  source 1..57: mol_type = other DNA
acactctttccctacacgacgctcttccgatctagatttctaagccacagaaaaaga

SEQ ID NO: 148 - DNA, 54 bp, synthetic construct
  source 1..54: mol_type = other DNA
gtgactggagttcagacgtgtgctcttccgatcttggtgaccagcattttatgg

SEQ ID NO: 149 - DNA, 54 bp, synthetic construct
  source 1..54: mol_type = other DNA
acactctttccctacacgacgctcttccgatctaatggaagatccagagaaagc

SEQ ID NO: 150 - DNA, 56 bp, synthetic construct
  source 1..56: mol_type = other DNA
gtgactggagttcagacgtgtgctcttccgatcttgctactccagtgaacacacaa

SEQ ID NO: 151 - DNA, 53 bp, synthetic construct
  source 1..53: mol_type = other DNA
acactctttccctacacgacgctcttccgatctgctggggagagaaaaggaaa

SEQ ID NO: 152 - DNA, 54 bp, synthetic construct
  source 1..54: mol_type = other DNA
gtgactggagttcagacgtgtgctcttccgatctgactgtgcaaatgcttctcg

SEQ ID NO: 153 - DNA, 53 bp, synthetic construct
  source 1..53: mol_type = other DNA
acactctttccctacacgacgctcttccgatctaggctattggtcaaggcaag

SEQ ID NO: 154 - DNA, 54 bp, synthetic construct
  source 1..54: mol_type = other DNA
gtgactggagttcagacgtgtgctcttccgatctacggctgacaaaagaagtcc

SEQ ID NO: 155 - DNA, 53 bp, synthetic construct; identical to SEQ ID NO: 151
  source 1..53: mol_type = other DNA

SEQ ID NO: 156 - DNA, 54 bp, synthetic construct; identical to SEQ ID NO: 152
  source 1..54: mol_type = other DNA

SEQ ID NO: 157 - DNA, 53 bp, synthetic construct
  source 1..53: mol_type = other DNA
acactctttccctacacgacgctcttccgatctgccagctaacatgggtcatt

SEQ ID NO: 158 - DNA, 53 bp, synthetic construct
  source 1..53: mol_type = other DNA
gtgactggagttcagacgtgtgctcttccgatctgccagaatcgagcactgag

SEQ ID NO: 159 - DNA, 53 bp, synthetic construct
  source 1..53: mol_type = other DNA
acactctttccctacacgacgctcttccgatctccttggcttgctatttggag

SEQ ID NO: 160 - DNA, 54 bp, synthetic construct
  source 1..54: mol_type = other DNA
gtgactggagttcagacgtgtgctcttccgatctggttccctcaaaacctcctc

SEQ ID NO: 161 - DNA, 53 bp, synthetic construct
  source 1..53: mol_type = other DNA
acactctttccctacacgacgctcttccgatcttagccactgtcctggcttct

SEQ ID NO: 162 - DNA, 54 bp, synthetic construct
  source 1..54: mol_type = other DNA
gtgactggagttcagacgtgtgctcttccgatctgcttcccagtgacacacaga

SEQ ID NO: 163 - DNA, 52 bp, synthetic construct
  source 1..52: mol_type = other DNA
acactctttccctacacgacgctcttccgatctgctctccacctcgatgtca

SEQ ID NO: 164 - DNA, 52 bp, synthetic construct
  source 1..52: mol_type = other DNA
gtgactggagttcagacgtgtgctcttccgatctatccggtgctgcagtgag

SEQ ID NO: 165 - DNA, 53 bp, synthetic construct
  source 1..53: mol_type = other DNA
acactctttccctacacgacgctcttccgatctttgctgtgtcttccttctgc

SEQ ID NO: 166 - DNA, 54 bp, synthetic construct
  source 1..54: mol_type = other DNA
gtgactggagttcagacgtgtgctcttccgatctttggtgggactgatgatgaa

SEQ ID NO: 167 - DNA, 54 bp, synthetic construct
  source 1..54: mol_type = other DNA
acactctttccctacacgacgctcttccgatctcagaagtggttgatgaattgc

SEQ ID NO: 168 - DNA, 54 bp, synthetic construct
  source 1..54: mol_type = other DNA
gtgactggagttcagacgtgtgctcttccgatctgaaaccatttgggctttcct

SEQ ID NO: 169 - DNA, 56 bp, synthetic construct
  source 1..56: mol_type = other DNA
acactctttccctacacgacgctcttccgatctttgctgagctcttatatgtgcaa

SEQ ID NO: 170 - DNA, 54 bp, synthetic construct
  source 1..54: mol_type = other DNA
gtgactggagttcagacgtgtgctcttccgatctttctcttgccctgaccattc

SEQ ID NO: 171 - DNA, 53 bp, synthetic construct
  source 1..53: mol_type = other DNA
acactctttccctacacgacgctcttccgatctagactcatagctggggctga

SEQ ID NO: 172 - DNA, 55 bp, synthetic construct
  source 1..55: mol_type = other DNA
gtgactggagttcagacgtgtgctcttccgatctccggatgattctcctacttcc

SEQ ID NO: 173 - DNA, 53 bp, synthetic construct
  source 1..53: mol_type = other DNA
acactctttccctacacgacgctcttccgatctcaaggtctgaggctcgaatc

SEQ ID NO: 174 - DNA, 54 bp, synthetic construct
  source 1..54: mol_type = other DNA
gtgactggagttcagacgtgtgctcttccgatctagagcaaaccttggcattgt

SEQ ID NO: 175 - DNA, 53 bp, synthetic construct
  source 1..53: mol_type = other DNA
acactctttccctacacgacgctcttccgatcttgagctcatttccaccagaa

SEQ ID NO: 176 - DNA, 54 bp, synthetic construct
  source 1..54: mol_type = other DNA
gtgactggagttcagacgtgtgctcttccgatctatgggaaatacgggcttagg

SEQ ID NO: 177 - DNA, 53 bp, synthetic construct
  source 1..53: mol_type = other DNA
acactctttccctacacgacgctcttccgatcttcagttttgcccgatctcct

SEQ ID NO: 178 - DNA, 54 bp, synthetic construct
  source 1..54: mol_type = other DNA
gtgactggagttcagacgtgtgctcttccgatctcccatggtgcctgactaaca

SEQ ID NO: 179 - DNA, 55 bp, synthetic construct
  source 1..55: mol_type = other DNA
acactctttccctacacgacgctcttccgatcttggctttccaaatcagtgggtc

SEQ ID NO: 180 - DNA, 57 bp, synthetic construct
  source 1..57: mol_type = other DNA
gtgactggagttcagacgtgtgctcttccgatctctcataagcttagaccaacaagc

SEQ ID NO: 181 - DNA, 53 bp, synthetic construct
  source 1..53: mol_type = other DNA
acactctttccctacacgacgctcttccgatctgctgagggccatctgaaaca

SEQ ID NO: 182 - DNA, 54 bp, synthetic construct
  source 1..54: mol_type = other DNA
gtgactggagttcagacgtgtgctcttccgatctagggtgccttcatggttagc

SEQ ID NO: 183 - DNA, 55 bp, synthetic construct
  source 1..55: mol_type = other DNA
acactctttccctacacgacgctcttccgatctctgtggtggaaatgctcccaag

SEQ ID NO: 184 - DNA, 56 bp, synthetic construct
  source 1..56: mol_type = other DNA
gtgactggagttcagacgtgtgctcttccgatctcactctcaggaagacaagcatc

SEQ ID NO: 185 - DNA, 53 bp, synthetic construct; identical to SEQ ID NO: 177
  source 1..53: mol_type = other DNA

SEQ ID NO: 186 - DNA, 54 bp, synthetic construct; identical to SEQ ID NO: 178
  source 1..54: mol_type = other DNA

SEQ ID NO: 187 - DNA, 23 bp, unidentified
  source 1..23: mol_type = other DNA
gtggggaaggggcccccaagagg

SEQ ID NO: 188 - DNA, 23 bp, unidentified
  source 1..23: mol_type = other DNA
ggtgggatggggtccccaagtgg

SEQ ID NO: 189 - DNA, 23 bp, unidentified
  source 1..23: mol_type = other DNA
ggtggggagcggccccccagtgg

SEQ ID NO: 190 - DNA, 23 bp, unidentified
  source 1..23: mol_type = other DNA
agtggggaggcgccctcaagtgg

SEQ ID NO: 191 - DNA, 22 bp, unidentified
  source 1..22: mol_type = other DNA
gtggggagtggcccccaagagg

SEQ ID NO: 192 - DNA, 22 bp, unidentified
  source 1..22: mol_type = other DNA
gtggggagcggccccccagtgg

SEQ ID NO: 193 - DNA, 23 bp, unidentified
  source 1..23: mol_type = other DNA
atgaggaagcgacccccaagagg

SEQ ID NO: 194 - DNA, 23 bp, unidentified
  source 1..23: mol_type = other DNA
ggtgagtgagtgtgtgcgtgtgg

SEQ ID NO: 195 - DNA, 23 bp, unidentified
  source 1..23: mol_type = other DNA
agtgagtgagtgtgtgtgtgggg

SEQ ID NO: 196 - DNA, 23 bp, unidentified
  source 1..23: mol_type = other DNA
tgtgagtaagtgtgtgtgtgtgg

SEQ ID NO: 197 - DNA, 23 bp, unidentified
  source 1..23: mol_type = other DNA
ggtgagtgtgtgtgtgcatgtgg

SEQ ID NO: 198 - DNA, 23 bp, unidentified
  source 1..23: mol_type = other DNA
gaagtattcattatagtcaaggg

SEQ ID NO: 199 - DNA, 23 bp, unidentified
  source 1..23: mol_type = other DNA
gaagcattcattatagtcaaagg

SEQ ID NO: 200 - DNA, 23 bp, unidentified
  source 1..23: mol_type = other DNA
ggcactgcggctggaggtggggg

SEQ ID NO: 201 - DNA, 23 bp, unidentified
  source 1..23: mol_type = other DNA
tgcactgcggccggaggaggtgg

SEQ ID NO: 202 - DNA, 23 bp, unidentified
  source 1..23: mol_type = other DNA
ggcacgacggctggaggtggggg

SEQ ID NO: 203 - DNA, 23 bp, unidentified
  source 1..23: mol_type = other DNA
ggcatcacggctggaggtggagg

SEQ ID NO: 204 - DNA, 25 bp, synthetic construct
  source 1..25: mol_type = other DNA
caccgtctgcttctccagccctggc

SEQ ID NO: 205 - DNA, 25 bp, synthetic construct
  source 1..25: mol_type = other DNA
aaatgccagggctggagaagcagac

SEQ ID NO: 206 - DNA, 25 bp, synthetic construct
  source 1..25: mol_type = other DNA
caccggatgttccaatcagtacgca

SEQ ID NO: 207 - DNA, 25 bp, synthetic construct
  source 1..25: mol_type = other DNA
aaattgcgtactgattggaacatcc

SEQ ID NO: 208 - DNA, 53 bp, synthetic construct
  source 1..53: mol_type = other DNA
acactctttccctacacgacgctcttccgatctgcctagaaaggcatggatga

SEQ ID NO: 209 - DNA, 54 bp, synthetic construct
  source 1..54: mol_type = other DNA
gtgactggagttcagacgtgtgctcttccgatctagcccctgtctaggaaaagc

SEQ ID NO: 210 - DNA, 53 bp, synthetic construct
  source 1..53: mol_type = other DNA
acactctttccctacacgacgctcttccgatctaagttgcccagagtcaagga

SEQ ID NO: 211 - DNA, 54 bp, synthetic construct
  source 1..54: mol_type = other DNA
gtgactggagttcagacgtgtgctcttccgatctcccaggtgctgacgtaggta

SEQ ID NO: 212 - DNA, 20 bp, unidentified
  source 1..20: mol_type = other DNA
ccactacgaccggattggtg

SEQ ID NO: 213 - DNA, 20 bp, unidentified
  source 1..20: mol_type = other DNA
cggctacggaatttaggaga

SEQ ID NO: 214 - DNA, 20 bp, unidentified
  source 1..20: mol_type = other DNA
ttcatacgaagaaggcagcc

(19) United States
(12) Patent Application Publication
(10) Pub. No.: US 2025/0041449 A1
(43) Pub. Date: Feb. 6, 2025

(54) BASE EDITOR AND USE THEREOF

(71) Applicants: SEOUL NATIONAL UNIVERSITY R&DB FOUNDATION, Seoul (KR); IUCF-HYU (INDUSTRY-UNIVERSITY COOPERATION FOUNDATION HANYANG UNIVERSITY), Seoul (KR); KOREA UNIVERSITY RESEARCH AND BUSINESS FOUNDATION, Seoul (KR)

(72) Inventors: Jeong Hun KIM, Seoul (KR); Jae-Sung WOO, Seoul (KR); Sang Su BAE, Seoul (KR)

(73) Assignees: SEOUL NATIONAL UNIVERSITY R&DB FOUNDATION, Seoul (KR); IUCF-HYU (INDUSTRY-UNIVERSITY COOPERATION FOUNDATION HANYANG UNIVERSITY), Seoul (KR); KOREA UNIVERSITY RESEARCH AND BUSINESS FOUNDATION, Seoul (KR)

(21) Appl. No.: 18/292,048

(22) PCT Filed: Jul. 26, 2022
(86) PCT No.: PCT/KR2022/010989

§ 371 Date: Jan. 25, 2024

(30) Foreign Application Priority Data
Jul. 26, 2021 (KR) 10-2021-0097893

(51) Int. Cl. A61K 48/00; C12N 9/22; C12N 9/78; C12N 15/11; C12N 15/90

(52) U.S. Cl. CPC A61K 48/0058; C12N 9/22; C12N 9/78; C12N 15/11; C12N 15/90

(57) ABSTRACT

Provided are a base editor, specifically, a base editor in the form of a ribonucleoprotein (RNP) complex, and uses in gene correction in vivo using the same. When using the base editor in the form of an RNP complex of the present disclosure, target genes can be effectively corrected by reducing off-target effects.

### TECHNICAL FIELD

The present disclosure relates to a base editor, for example, a base editor in the form of a ribonucleoprotein (RNP) complex, and uses in gene correction in vivo using the same. When using the base editor in the form of an RNP complex of the present disclosure, target genes can be effectively corrected by reducing off-target effects.

### BACKGROUND ART

The gene-editing technology utilizing the CRISPR/Cas9 system has brought about a tremendous advancement in current genetic therapy. These gene-editing tools typically induce DNA double-strand breaks (DSBs). Then, living cells recognize these DSBs as severe damage, triggering systems to initiate repair. Specifically, cells engage in two pathways to restore the cleaved DNA strands: Non-Homologous End Joining (NHEJ), which induces diverse genetic sequence modifications as it operates swiftly without relying on a repair template, and Homologous-Directed Repair (HDR), allowing precise repair via the use of a template strand. In general, NHEJ is utilized for gene removal, and HDR is employed for gene correction or insertion purposes.

Meanwhile, correction through HDR is known to be highly inefficient, particularly DNA cleavage by Cas9 nuclease often causes unwanted DNA insertion or deletion (indel) in the target site. To compensate these limitations, recent approaches involve removing the cleavage function of the CRISPR/Cas system and using only its ability to recognize specific DNA sequences. The approaches involve fusing a catalytically impaired Cas9 protein (dead Cas9; dCas9), devoid of its cleavage ability, with various genetic regulatory factors. Particularly, the emergence of a fourth-generation gene editing technology, known as base editors (referred to as ‘base editors’), allows for the correction or alteration of specific sequences, enabling either gene disruption or the conversion of genes into desired traits.

There are broadly two types of base editors used. In short, there are a cytidine base editor (CBE) and an adenine base editor (ABE), the CBE capable of finding only cytosine (C) in DNA sequences and replacing it to thymine (T) by combining cytidine deaminase with the deactivated Cas9 (dCas9) lacking the double-stranded DNA cutting function of CRISPR/Cas9 or Cas9 nickase (nCas9) lacking only the single-stranded DNA cutting function, and the ABE capable of replacing adenine (A) to guanine (G) by combining adenine deaminase therewith. These base editors rarely produce DNA double-strand breaks (DSBs) and do not require a donor DNA template. Therefore, base editors are widely used in a wide range of research fields, including plant genome engineering, mouse zygote engineering, and biomedical applications.

However, similar to the CRISPR nuclease, base editors show off-target effects throughout the entire single guide RNA (sgRNA)-dependent genome. ABE was also found to catalyze the conversion of cytosine in addition to adenine located in the target sequence motif. In addition, CBE induces promiscuous DNA deamination in the genome, independent of sgRNA, and both CBE and ABE lead to promiscuous RNA deamination across the entirety of the transcriptome. To address these issues, Cas9 effector engineering technique was used or cytidine/adenine deaminase was modified, but the problems have not been completely solved.

Another approach to reduce off-target effects is by altering the means of delivering base editor. When employing intracellular delivery of bacterial plasmids or viral vectors encoding base editors and sgRNAs, exogenous base editors are continuously produced within cells and intracellular concentration is difficult to control, typically results in elevated off-target editing rates, consequently leading to accumulated off-target effects.

To reduce off-target effects, in contrast, base editor-sgRNA-RNP complexes have been utilized, but currently, the RNP system is not widely used in biological fields and has a difficulty in manufacturing base editor proteins with high purity and yield.

### DISCLOSURE

### Technical Problem

An object of the present disclosure is to provide a base editor, specifically, a base editor in the form of a ribonucleoprotein (RNP) complex.

Another object of the present disclosure is to provide gene correction in vivo using a base editor in the form of an RNP complex, and in particular, to effectively correct target genes associated with retinal dysfunction diseases, such as retinal degenerative diseases, by reducing off-target effects.

Still another object of the present disclosure is to provide a system or method for purifying a base editor with high purity.

### Technical Solution

In one general aspect, there is provided a fusion protein comprising a Cas9 domain; and an adenine deaminase domain or a cytidine deaminase domain.

In another general aspect, there is provided a complex comprising: (i) a fusion protein comprising a Cas9 domain; and an adenine deaminase domain or a cytidine deaminase domain; and (ii) a guide RNA (gRNA), wherein the gRNA is bound to the Cas9 domain of the fusion protein.

In another general aspect, there is provided a pharmaceutical composition for preventing or treating diseases or disorders, particularly, retinal dysfunction, comprising: (i) a fusion protein comprising Cas9 domain; and an adenine deaminase domain or a cytidine deaminase domain; and (ii) a guide RNA (gRNA).

In an embodiment, the fusion protein may further comprise an uracil glycosylase inhibitor (UGI) domain. The gRNA may be a single guide RNA (sgRNA).

In an embodiment, the Cas9 domain may be nCas9, which cleaves the nucleotide target strand in a nucleotide duplex, or the Cas9 domain may recognize a protospacer adjacent motif (PAM) of the target nucleic acid sequence, and the PAM may be NGG or NG.

In an embodiment, the retinal dysfunction may be caused by a genetic mutation, such as a point mutation. The retinal dysfunction may be a retinal degenerative disease; retinitis pigmentosa; retinitis pigmentosa; angiopathy; Drusen; lebers congenital amaurosis; hereditary or acquired macular degeneration; age-related macular degeneration (AMD); Best disease; retinal detachment; cerebral atrophy; choroidal defect; pattern dystrophy; retinal pigment epithelium (RPE) dystrophy; Stargardt disease; selection from retinal pigment epithelium (RPE) and retinal damage caused by any of light, laser, infection, radiation, neovascularization or traumatic injury; retinal dysplasia; color blindness; choroideremia; myopic choroidal neovascularization; nodular choroidal angiopathy; central serous chorioretinopathy; macular hole; macular dystrophy; diabetic retinopathy; retinal arteriovenous occlusion; hypertensive retinopathy; retinal aortic aneurysm; ophthalmic ischemia syndrome; retinopathy of prematurity; acute retinal necrosis; cytomegalovirus retinitis; toxoplasma retinochoroiditis; syphilitic chorioretinitis; retinal detachment; or retinoblastoma.

In a specific embodiment, the gRNA may comprise a sequence of contiguous nucleotides complementary to a target sequence associated with the retinal dysfunction, and the target sequence may comprise a point mutation associated with the retinal dysfunction.

In a specific embodiment, the fusion protein or complex may correct the point mutation. The point mutation may comprise a T to C point mutation and deamination of the mutant C base may result in a sequence not associated with the retinal dysfunction; or the point mutation may comprise a G to A point mutation, and deamination of the mutant A base may result in a sequence not associated with the retinal dysfunction.

In another general aspect, there is provided a purification method of a fusion protein, comprising:
(a) expressing a fusion protein in a cell, the fusion protein containing a Cas9 domain; and an adenine deaminase domain or a cytidine deaminase domain, wherein the fusion protein further includes an affinity tag;
(b) dissolving the fusion protein expressed in Step (a) to produce a lysate;
(c) performing primary purification on the lysate of Step (b) by affinity chromatography;
(d) performing secondary purification on the fusion protein purified in Step (c) by affinity chromatography; and
(e) performing third purification on the fusion protein purified in Step (d) by size exclusion chromatography.

### Advantageous Effects

Base editing via delivery of the base editor protein and gRNA, particularly the ABE/CBE RNP complex, according to the present disclosure, results in reduced off-target effects in both DNA and RNA compared to base editing via delivery of plasmid-encoded ABE/CBE. Due to the reduced off-target effect of RNP complex-mediated base editing, the ABE/CBE RNP complex of the present disclosure is expected to be very beneficial, especially in therapeutic applications.

In addition, it was confirmed that in vivo gene correction in retinal degeneration 12 (rd12) model mice has been successfully induced using NG-ABEmax RNP, the ABE RNP complex of the present disclosure. Thus, the base editor protein or RNP thereof according to the present disclosure may be particularly useful in the field of treatment related to ocular diseases, such as the treatment of retinal dysfunction such as retinal degenerative disease.

### DESCRIPTION OF DRAWINGS

FIG. 1 illustrates the purification process and results of ABE/CBE proteins in human cell expression system:

FIG. 1a illustrates a schematic diagram of a purification process for a base editor;

FIG. 1b illustrates the edited base editor with peak fractions of ABEmax or AncBE4max collected by size exclusion chromatography, concentrated and analyzed by SDS-PAGE;

FIG. 1c illustrates the percentage of sequence reads (%) for specific edited nucleotides converted from target C in HEK293T cells after RNP- and plasmid-mediated CBE delivery with or without UGI overexpression; and

FIG. 1d illustrates viability of HEK293T cells after RNP- and plasmid-mediated ABE/CBE delivery and GFP vector delivery, wherein the viability was determined using the CCK-8 assay.

FIG. 2 illustrates constructs for base editor expression and purification showing characteristics of RNP- and plasmid-mediated ABE and CBE DNA editing: Specifically, schematic diagrams of pEX-FlagR-ABEmax and pEX-FlagR-BE4max plasmids used to express base editor proteins are shown (ABEmax and AncBE4max were used in the present disclosure), wherein the hatched part with [symbol] represents the linker peptide.

FIG. 3 illustrates the characteristics of RNP- and plasmid-mediated ABE and CBE DNA editing:

FIGS. 3a and 3c illustrate the extent of base editing in HEK293T cells after RNP- and plasmid-mediated ABE (a) or CBE (c) delivery at 19 or 8 genomic sites, respectively; expressed as the percentage of sequencing reads containing an A conversion (for ABE) or C conversion (for CBE) at positions 2-11 among the total sequencing reads, wherein bars represent mean values, and error bars represent Standard Error of the Mean (s.e.m) of three independent biological replicates; and

FIGS. 3b and 3d illustrate indels in HEK293T cells after RNP- and plasmid-mediated ABE (b) or CBE (d) delivery at 19 or 8 genomic sites, respectively; expressed as the percentage of sequencing reads with indels among the total sequencing reads, wherein dots represent the average of three independent biological replicates for each target, and error bars represent s.e.m. of the values indicated by the dots.

FIG. 4 illustrates RNP- and plasmid-mediated editing characteristics and human endogenous DNA target sites:

FIGS. 4a and 4b illustrate the number of base conversions in the ABE editing allele, wherein bars represent the ratio of the number of reads containing single (7) [symbol] , double ( [symbol] ), or triple ( [symbol] ) A conversions to the total number of reads containing A conversions; in FIG. 4a, the most common sequences (positions 1-20) of ABE site 5 are presented, accompanied by their respective frequencies indicated on the right, and hatched nucleotides represent edited sequences;

FIGS. 4c and 4d illustrate the number of base conversions in CBE edited alleles at different target sites, wherein bars represent the ratio of the number of reads containing single ( [symbol] ), double ( [symbol] ), or triple ( [symbol] ) C conversions and the total number of reads containing C conversions;

In FIGS. 4a-4d, error bars represent s.e.m. of three independent biological replicates;

FIG. 4e illustrates the ABE editing efficiency of HEK293T cells at different time points after transfection of the ABE-encoding plasmid;

FIG. 4f illustrates the number of base conversions in the ABE editing allele at different time points after transfection of the ABE-encoding plasmid, wherein error bars represent s.e.m. of two independent biological replicates; and

FIGS. 4g and 4h illustrate the results of ABE abundance analysis in HEK293T cells at different time points after transfection of ABE protein ( [symbol] ) or ABE encoding plasmid ( [symbol] ) in the absence of sgRNA; in FIG. 4g, each dot represents the ABE abundance relative to the maximum abundance obtained for a given delivery method; and in FIG. 4h, bars represent ABE abundance relative to that obtained from plasmid-mediated expression 6 hours after transfection; wherein dots (shown in g) and bars (shown in h) represent the average of two independent biological replicates.

FIG. 5 illustrates the results of Western blot analysis to estimate relative ABE abundance:

FIG. 5a illustrates representative images of Western blots used to estimate ABE abundance in HEK293T cells at different time points after delivery of ABE protein or ABE encoding plasmid in the absence of sgRNA;

FIG. 5b illustrates the results of ABE abundance analysis in HEK293T cells at different time points after transfection of ABE protein ( [symbol] ) or ABE encoding plasmid ( [symbol] ) in the presence of sgRNA; in the top graph, each dot represents the ABE abundance relative to the maximum abundance obtained for a given delivery method; and in the bottom graph, bars represent ABE abundance relative to that obtained from plasmid-mediated expression 24 hours after transfection; wherein the dots in the upper graph and the bars in the lower graph represent the average of two independent biological replicates; and

FIG. 5c illustrates representative images of Western blots used to estimate ABE abundance in (b).

FIG. 6 illustrates optimization results of mRNA-mediated ABE editing and chemically synthesized sgRNA-mediated ABE editing:

FIG. 6a illustrates the extent of base editing in HEK293T cells after delivery of 3.0 μg of ABE encoding mRNA using different doses of in vitro transcribed sgRNA, wherein bars represent the average of two independent biological replicates; and

FIG. 6b represents the degree of base editing in HEK293T cells after delivery of ABE RNP or ABE encoding mRNA using chemically synthesized sgRNA targeting HEK_site 2, wherein bars represent mean values and error bars represent s.e.m. of four independent biological replicates.

FIG. 7 illustrates delivery-dependent variation of ABE-mediated DNA off-target effects:

FIG. 7a illustrates off-target DNA base editing in HEK293T cells after delivery of ABE RNP, ABE-encoding mRNA and ABE-encoding plasmid, expressed with A-to-G editing efficiency (top panel) and ratio of off-target to on-target A-to-G editing efficiency (bottom panel), wherein bars represent mean values and error bars represent s.e.m. of three independent biological replicates;

FIG. 7b illustrates a schematic diagram of orthogonal R-loop analysis; and

FIGS. 7c and 7d illustrate the frequency of sgRNA independent off-target DNA editing detected by orthogonal R-loop analysis, wherein ABE RNP or ABE-encoding plasmid (FIG. 7c) and CBE RNP or CBE-encoding plasmid (FIG. 7d) were used, respectively, for co-transfection employing the dSaCas9-encoding plasmid together with sgRNA targeting HEK_site 2, and SaCas9 sgRNA targeting R-loop 5 or 6 for each R-loop, where bars represent mean values and error bars represent s.e.m. of three independent biological replicates.

FIG. 8 illustrates delivery-dependent variation of ABE-mediated DNA off-target effects:

FIG. 8a illustrates the extent of off-target RNA base editing in HEK293T cells after delivery of ABE RNP, ABE-encoding mRNA, and ABE-encoding plasmid in the presence of sgRNA, indicating the efficiency of A-to-I mRNA editing, and delivery of a plasmid encoding green fluorescent protein (GFP) was used as a control, wherein bars represent mean values and error bars represent s.e.m. of four independent biological replicates; and

FIG. 8b illustrates the off-target RNA base editing in HEK293T cells after delivery of ABE protein and ABE-encoding plasmid in the absence of sgRNA, indicating the efficiency of A-to-I mRNA editing, wherein bars represent mean values and error bars represent s.e.m. of four independent biological replicates.

FIG. 9 illustrates the activity of RNP-mediated DNA editing using chemically synthesized sgRNA or NG-ABEmax for in vivo DNA editing:

FIG. 9a illustrates a schematic diagram of ABEmax RNP delivery into mice via subretinal injection, where RNPs (circles filled with dots) were encapsulated in cationic lipid nanoparticles;

FIG. 9b illustrates the DNA sequences of Fah, Vegfa, and Nr2e3 used to demonstrate the efficiency of ABE RNP-mediated DNA editing in vivo in normal mice; and

FIG. 9c illustrates the degree of base editing in three genomic regions of HEK293T cells after NG-ABEmax RNP delivery, with the percentage of sequencing reads containing A conversion at positions 2-11 among the total sequencing reads, wherein bars represent mean values and error bars represent s.e.m. of three independent biological replicates.

FIG. 10 illustrates the extent of NG-ABEmax RNP-mediated correction of disease-related mutations in rd12 mice:

FIG. 10a illustrates the NG-ABEmax RNP-mediated correction strategy of nonsense mutations in Rpe65 in rd12 mice;

FIG. 10b illustrates the efficiency of NG-ABEmax RNP-mediated mutation correction in rd12 mEFs using various sgRNAs, where the control was NG-ABEmax without sgRNA, wherein bars represent the average of two independent replicates, gX19 and gX20 represent in vitro transcribed sgRNAs containing 19-nt and 20-nt spacers, respectively, with a mismatched 5′G, X19 and X20 represent chemically synthesized sgRNA containing a 19-nt spacer and a 20-nt spacer, respectively, and gX19+CIP indicates the treatment involving both gX19 and calf intestinal alkaline phosphatase (CIP, NEB);

FIG. 10c illustrates a schematic diagram of NG-ABEmax RNP-mediated treatment performed on rd12 mice via subretinal injection, wherein RNPs (circles filled with dots) were encapsulated in cationic lipid nanoparticles;

FIG. 10d illustrates a representative confocal image of RPE from rd12 mice at 6 hours after injection, with a scale bar of 10 μm;

FIG. 10e illustrates the efficiency of NG-ABEmax RNP-mediated mutation correction in rd12 mice (n=8), where genomic DNA obtained from RPE of eyes, either injected or not injected with NG-ABEmax RNPs, was analyzed to determine the efficiency of targeted A-to-G editing, wherein bars represent mean values and error bars represent s.e.m. of eight independent replicates. ***P<0.001 by Mann-Whitney test;

FIG. 10f illustrates the relative expression of Rpe65 mRNA in RPE from rd12 mice injected with NG-ABEmax RNPs (n=4); and

FIG. 10g illustrates a representative confocal microscopy image of RPE from rd12 mice 5 weeks after injection, with a scale bar of 10 μm. ***P<0.001 (Kruskal-Wallis test with Dunn's multiple comparison test), Non-target indicates Non-target rd12 mice injected with NG-ABEmax RNP containing non-targeting sgRNA; Target indicates Target rd12 mouse injected with NG-ABEmax RNP containing targeting sgRNA.

FIG. 11 illustrates the results of in vivo NG-ABEmax RNP treatment to correct disease-related mutations in rd12 mice:

FIG. 11a illustrates a representative confocal image of RPE from rd12 mice 72 hours after NG-ABEmax RNP injection, with a scale bar of 10 μm; and

FIG. 11b illustrates representative sequencing results from DNA isolated from RPE from eyes of juvenile and adult rd12 mice injected with NG-ABEmax and targeting sgRNA, where the mutated T nucleotide in rd12 mice and the correction efficiency at this position are indicated by hatching [symbol] .

### BEST MODE

Fusion Proteins, gRNAs and Complexes Thereof

The present disclosure provides a fusion protein comprising a Cas9 domain; and an adenine deaminase domain or a cytidine deaminase domain.

In an embodiment, the present disclosure provides a complex comprising: (i) a fusion protein comprising a Cas9 domain; and an adenine deaminase domain or a cytidine deaminase domain; and (ii) a guide RNA (gRNA), wherein the gRNA is bound to the Cas9 domain of the fusion protein.

As used herein, the term “Cas9” or “Cas9 nuclease” refers to an RNA-guided nuclease comprising the Cas9 protein, or fragments thereof (for example, a protein comprising an active, inactive, or partially active DNA cleavage domain of Cas9, such as a Cas nickase, and/or a gRNA binding domain of Cas9). Cas9 is an enzyme that is bound to the guide RNA to induce cleavages or modifications in the sequence or location of the target gene or nucleic acid, and comprises an HNH domain capable of cleaving the nucleic acid strand that is complementary to the guide RNA, a RuvC domain capable of cleaving the nucleic acid strand that binds non-complementarily to the guide RNA, a REC that recognizes the target, and a PI domain that identifies the PAM sequence. The specific sequence and structure of Cas9 are well known to those skilled in the art (for example, [Ferretti et al., Proc. Natl. Acad. Sci. U.S.A. 98:4658-4663 (2001)]; [Deltcheva E. et al., Nature 471:602-607 (2011)]; and [Jinek M. et al., Science 337:816-821 (2012)], the entire contents of each of which are incorporated herein by reference.

The inactive protein of Cas9 may be interchangeably referred to as “dCas9” protein (i.e., nuclease-“dead” Cas9). Methods for producing Cas9 proteins (or fragments thereof) having inactive DNA cleavage domains are known (for example, [Jinek et al., Science. 337:816-821 (2012)]; [Qi et al., “Repurposing CRISPR as an RNA-Guided Platform for Sequence-Specific Control of Gene Expression” (2013) Cell. 28; 152 (5):1173-83], the entire contents of each of which are incorporated herein by reference).

As used herein, the term “Cas9 nickase” or “nCas9” refers to the Cas9 protein capable of cleaving only one strand of a duplex nucleic acid molecule (for example, a duplex DNA molecule). Accordingly, the Cas9 domain of the present disclosure may be nCas9, which cleaves the nucleotide target strand in a nucleotide duplex.

In an embodiment, a protein comprising a fragment of Cas9 is provided. For example, the protein comprises one of the two Cas9 domains: (1) gRNA binding domain of Cas9; or (2) DNA cleavage domain of Cas9. In an embodiment, proteins comprising Cas9 or fragments thereof are referred to as “Cas9 variants”. A Cas9 variant shares homology to Cas9, or a fragment thereof.

As used herein, the term “deaminase” or “deaminase domain” refers to a protein or enzyme that catalyzes the deamination reaction. In an embodiment, the deaminase or deaminase domain is adenine deaminase; or cytidine deaminase.

Adenine deaminase is as defined in the art (see, e.g., U.S. Pat. No. 10,113,163, etc.) and catalyzes the hydrolytic deamination of adenine or adenosine. In an embodiment, adenine deaminase catalyzes the hydrolytic deamination of adenosine or deoxyadenosine to inosine or deoxyinosine, respectively. In some embodiments, adenine deaminase converts A to G in the sense strand of the target nucleic acid and converts T to C in the anti-sense strand of the target nucleic acid.

Similarly, cytidine deaminase catalyzes the hydrolytic deamination of cytidine or deoxycytidine to uridine or deoxyuridine. In some embodiments, cytidine deaminase converts C to T in the sense strand of the target nucleic acid and converts G to A in the anti-sense strand of the target nucleic acid.

In an embodiment, the deaminase or deaminase domain is a naturally-occurring deaminase from an organism, such as a human, chimpanzee, gorilla, monkey, cow, dog, rat, or mouse. In an embodiment, the deaminase or deaminase domain is a variant of a naturally-occurring deaminase from an organism that does not occur in nature.

As used herein, the term “fusion protein” refers to a hybrid polypeptide comprising protein domains from at least two different proteins. One protein is located either at the amino-terminal (N-terminal) portion of the fusion protein or at the carboxy-terminal (C-terminal) portion of the protein, and is thus capable of forming “amino-terminal fusion protein” or “carboxy-terminal fusion protein”, respectively. The protein may comprise different domains of a nucleic acid editing protein, such as a nucleic acid binding domain (for example, the gRNA binding domain of Cas9 that directs the binding of the protein to a target site) and a nucleic acid cleavage domain or a catalytic domain. In an embodiment, the fusion proteins of the present disclosure may exist in the form of a complex with a nucleic acid, such as RNA.

As used herein, the term “Base Editor (BE)”, sometimes also referred to as “NucleoBase Editor (NBD)”, represents a base editing tool derived from CRISPR gene editing, and works by replacing a single base, unlike existing genetic scissors that cut both strands of DNA. The base editor consists of Cas9 nickase (nCas9) that cuts one strand of DNA, and deaminase that decomposes adenine or cytosine. In this case, the term “base editor” as used herein refers to the Cas9 fusion protein described herein. Specifically, there are an adenine base editor (ABE) that combines adenine deaminase and a cytosine base editor (CBE) that combines cytosine deaminase, with dead Cas9 (dCas9) or nCas9 lacking the CRISPR/Cas9 double-stranded DNA cutting function. For example, in the case of CBE, when deaminase replaces cytosine (C) with uracil (U) in one strand of cut DNA, the base converted to uracil (U) becomes thymine (T) through the DNA repair process. Using base editors, genes may be deleted or transformed by correcting or replacing specific sequences.

In an embodiment, the adenine deaminase may originate from bacteria, such as E. coli, S. aureus, S. typhi, S. putrefaciens, H. influenzae, or C. crescentus. In an exemplary embodiment, adenine deaminase may be an engineered E. coli TadA deaminase engineered to be applied to TadA deaminase, such as E. coli TadA deaminase (ecTadA), truncated E. coli TadA deaminase, or deoxynucleotide, but is not limited thereto.

In one embodiment, cytidine deaminase is an apolipoprotein B mRNA-editing complex (APOBEC) family deaminase. In an embodiment, the deaminase is APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, APOBEC3H deaminase, or APOBEC4 deaminase. In an embodiment, the deaminase is activation-induced deaminase (AID). In an embodiment, the deaminase is lamprey CDA1 (pmCDA1) deaminase.

The types of adenine deaminase or cytidine deaminase described above are merely illustrative, and the scope of the present disclosure is not limited thereto, and it is interpreted to include all ranges capable of being changed or modified normally in the art.

In an embodiment, the base editor or fusion protein of the disclosure, particularly a cytosine base editor (CBE) comprising cytosine deaminase, may further comprise an uracil glycosylase inhibitor (UGI) domain.

As used herein, the term “uracil glycosylase inhibitor” or “UGI” refers to a protein capable of inhibiting an uracil-DNA glycosylase base-excision repair enzyme. The UGI domain usable herein may be a domain known in the art.

In an embodiment, the fusion protein of the present disclosure may be used without limitation as long as it is a fusion protein known in the art. Exemplary fusion proteins capable of being used in the present disclosure may be, but are not limited to, any of those listed in Tables 1 (CBE fusion protein) and 2 (ABE fusion protein) below.

TABLE 1

| | | | AncBE4max- |
|---|---|---|---|
| AncBE4max | enAsCas12a-BE | A3A-BE3 | P2A-GFP |
| BE1 | ScCas9-BE3 | eA3A-BE3 | CBE1 |
| | | | (rAPOBEC1- |
| | | | XTEN-dCas9- |
| | | | NLS) |
| BE2 | YE1-BE3 or | eA3A-HF1-BE3- | CBE2 |
| | YE1-BE4 | 2xUGI | (rAPOBEC1- |
| | | | XTEN-dCas9- |
| | | | UGI-NLS) |
| BE3 | EE-BE3 | eA3A-Hypa-BE3- | CBE3 |
| | | 2xUGI | (rAPOBEC1- |
| | | | XTEN-nCas9- |
| | | | UGI-NLS) |
| BE4 | YE2-BE3 | BE3 (hA3A-Y130F) | CBE4 |
| BE4max | YEE-BE3 | BE3 (hA3A-R128A) | CBE4-Gam |
| xCas9 (3.7)- | YFE-BE4max | Target-AID | S. aureaus |
| BE3 | | | CBE4 |
| VQR-BE3 | dCpf1-BE-YE | nCDA1-BE3 | S. aureaus |
| | | | BE4-Gam |
| EQR-BE3 | CP1012-CBE | Target-AID-NG | dCBE4 |
| VRER-BE3 | CP1028-CBE | hAID-BE3 | dCBE4-Gam |
| CBE-NG | HF-BE3 | BE4-PpAPOBEC1 | ABE-P48R |
| | | [R33A] | |
| Spy-mac | Sniper-BE3 | BE4-PpAPOBEC1 | ABE-P48R- |
| BE4max | | [H122A] | UGI |
| Sa-BE3 | SECURE-BE3 | BE4-RrA3F | |
| SaKKH-BE3 | SECURE-BE3 | BE4-AmAPOBEC1 | |
| SauriBE4ma | BE3-R132E | BE4-SsAPOBEC3B | |
| x | | | |
| dLbCpf1-BE | BE3- | | |
| | (W90F + R126E) | | |

TABLE 2

| ABEmax | SauriABEmax | CP1249-ABE |
|---|---|---|
| ABE7.10 | ABE8e | ABEmax-AW |
| VQR-ABE | SaABE8e | ABE8e (TadA-8e V106W) |
| VRQR-ABE | LbABE8e | ABE8.17-m |
| VRER-ABE | enAsABE8e | SECURE-ABE (K20A/R21A) |
| NG-ABE | ABE8s | SECURE-ABE (V82G) |
| xCas9 (3.7)- | NG-ABE8s | ABE7.10-F148A |
| ABE | | |
| Spy-mac | Sa-ABE8s | ABE4max-P2A-GFP |
| ABEmax | | |
| Sa-ABE | CP1012-ABE | dABE7.10 |
| | | (ecTadA (wt)-linker (32aa)- |
| | | ecTadA* (7.10)-linker (32aa)- |
| | | dCas9-NLS) |
| SaKKH-ABE | CP1028-ABE | ABE8eWQ |
| ScCas9-ABE | CP1041-ABE | ABE8eWA |

In another embodiment, the fusion protein of the present disclosure may be ABEmax or AncBE4max, such as ABEmax and AncBE4max expressed from pCMV_ABEmax (addgene no. 112095) and pCMV_AncBE4max (addgene no. 112094), respectively.

In another embodiment, the Cas9 domain of the fusion protein of the present disclosure recognizes a protospacer adjacent motif (BAM) of the target nucleic acid sequence, where the PAM may be NGG or NG, and may be various PAMs known in the art, for example, a PAM recognized by a Cas9 variant (for example, near PAM-less Cas9 variant (SpRY)) (e.g., see Russell T. Walton et al., Science 17-4-2020: Vol. 368, Issue 6488, pp. 290-296). Accordingly, the above-described fusion protein may target NG PAM. In an exemplary embodiment, the fusion protein targeting NG PAM may be one in which the Cas9 sequence of pCMV_ABEmax is replaced with the SpCas9-NG sequence of pX330-SpCas9-NG (addgene no. 117919).

As used herein, the term “guide RNA” or “gRNA” refers to a target gene- or nucleic acid-specific RNA, which binds to the CRISPR enzyme and guides the CRISPR enzyme to the target gene or nucleic acid. gRNA may exist as a complex of two or more RNAs, or as a single RNA molecule. gRNA that exists as a single RNA molecule may be referred to as a single-guide RNA (sgRNA), and “gRNA” is used interchangeably to refer to a guide RNA that exists as a single molecule or as a complex of two or more molecules. Typically, a gRNA, existing as a single RNA species, contains two domains: (1) a domain (crispr RNA; crRNA) that shares homology with the target nucleic acid (for example, specifies binding of the Cas9 complex to the target); and (2) a domain that binds to the Cas9 protein (transactivating RNA; tracrRNA).

In an embodiment, the fusion protein of the present disclosure and the gRNA may exist in the form of a complex, where the gRNA is able to be bound to the Cas9 domain. Here, the complex may be in the form of ribonucleoprotein (RNP).

As used herein, the term “RNP (ribonucleoprotein)” refers to a complex of RNA and protein. Among RNAs that exist in nature, most RNAs except tRNA exist in combination with proteins. Specific examples thereof include ribosomes, spliceosomes, messenger ribonucleoproteins (mRNA), CRISPR/CAS9, and the like. Ribosomes, the site of protein synthesis within cells, form a granular structure by combining RNA with dozens of types of neutral or weakly basic proteins. The RNA may be single guide RNA (sgRNA), crspr RNA (crRNA), or transactivating RNA (tracrRNA). Here, the RNA sequence may have a sequence complementary to the target gene sequence. In a specific embodiment, the gRNA of the present disclosure may include a sequence of contiguous nucleotides that are complementary to a target nucleic acid sequence associated with retinal dysfunction (for example, retinal degenerative disease, etc.). The target gene may be selected from various genes depending on the purpose. The protein herein may be a fusion protein, that is, a base editor.

Base editing by the fusion protein of the present disclosure, particularly in the form of RNP complex comprising the fusion protein and gRNA (hereinafter also referred to as “RNP complex”), exhibits reduced off-target effects. Further, base editing through the RNP complex of the present disclosure exhibits an editing pattern with a lower ratio of multiple base conversions to single base conversions compared to plasmid-encoded fusion protein (i.e., ABE or CBE), which is mainly because the ABE/CBE RNP complex of the present disclosure has a short lifespan in cells.

As used herein, the term “base editing” refers to a process in which a nucleotide base is modified when compared to an initial (e.g., wild-type) base at the same position. In base editing, adenine and cytidine deaminases remove amino groups from respective nucleotide targets thereof, resulting in conversion to inosine and uridine, respectively. During DNA repair or replication, inosine is recognized as guanine and uridine as thymine by the polymerase enzyme, converting A:T base pairs to G:C base pairs or C:G base pairs to T:A base pairs in edited double-stranded DNA.

As used herein, the term “base editing window” refers to any base that needs to be edited by a base editor, typically indicating a certain distance from any component of the editing system that is within the region accessible after binding of at least one component of the base editing system to the target DNA. Base editors that require a PAM sequence (for example, Cas9-containing editors) typically have base editing windows of 3, 4, 5, 6, 7, or more nucleotides that may be 13 to 16 or more nucleotides from the PAM sequence.

As used herein, the term “on-target” refers to the sequence or position within a target gene or nucleic acid to which a target-specific base editor binds in a complementary manner, and the term “off-target” as used herein refers to “target)” refers to a sequence or position within a target gene or nucleic acid where a target-specific base editor has partially complementary binding and induces base editing activity despite it not being the intended site.

The off-target is a sequence or position within a gene or nucleic acid that is not targeted by the target-specific base editor, or a nucleic acid sequence with less than 100% sequence homology to the nucleic acid sequence of the on-target. The nucleic acid sequence with less than 100% sequence homology to the nucleic acid sequence of the on-target is a nucleic acid sequence similar to the on-target nucleic acid sequence, which may contain one or more different base sequences or have one or more base sequences deleted.

### Uses of Fusion Protein or RNP Complex

The present disclosure provides uses of a fusion protein or RNP complex, specifically for use in in vivo gene editing. In an embodiment, the fusion protein or RNP complex of the present disclosure may provide correction of a genetic defect by deamidating a target nucleobase, e.g., an A residue or a C residue, e.g., correction of point mutation resulting in loss of function in the gene product. In an embodiment, the genetic defect is associated with a disease or disorder, such as retinal dysfunction, especially retinal degenerative disease.

Accordingly, the present disclosure provides a pharmaceutical composition for preventing or treating retinal dysfunction, particularly retinal degenerative disease, comprising: (i) a fusion protein comprising Cas9 domain; and an adenine deaminase domain or a cytidine deaminase domain; and (ii) guide RNA (gRNA). The fusion protein and gRNA may exist in the form of a ribonucleoprotein (RNP) complex.

In an embodiment, the target DNA sequence comprises a sequence associated with a disease or disorder, such as a point mutation associated with the disease or disorder. In an embodiment, the activity of the Cas9 protein, Cas9 fusion protein, or RNP complex corrects a point mutation. In an embodiment, the target DNA sequence comprises a G-A point mutation associated with a disease or disorder, wherein deamination of the mutant A base results in a sequence that is not associated with the disease or disorder. In another embodiment, the target DNA sequence comprises a T-C point mutation associated with a disease or disorder, wherein deamination of the mutant C base results in a sequence that is not associated with the disease or disorder.

In an embodiment, the target DNA sequence encodes a protein, wherein the point mutation is in a codon and results in a change in the amino acid encoded by the mutant codon compared to the wild-type codon. In an embodiment, deamination of mutant A or C results in a change in the amino acid encoded by the mutant codon. In an embodiment, deamination of mutant A or C results in a codon encoding a wild-type amino acid.

In a specific embodiment, the target DNA sequence may be associated with retinal dysfunction, particularly retinal degenerative disease. The retinal dysfunction, especially retinal degenerative disease, may be caused by a genetic mutation, specifically, a point mutation. Accordingly, the fusion protein or RNP complex of the present disclosure may act on a target sequence in which a gene mutation occurs in retinal dysfunction, such as retinal degenerative disease. In a specific embodiment, the RNP complex may contact a target DNA sequence, wherein the gRNA may comprise a sequence of contiguous nucleotides complementary to the target sequence associated with the retinal dysfunction, such as a retinal degenerative disease.

In the present disclosure, point mutations associated with retinal dysfunction, particularly retinal degenerative diseases, may include point mutations from T to C, and the deamination of the mutant C base by the fusion protein or RNP complex of the present disclosure may generate a sequence that is not associated with the retinal dysfunction, particularly retinal degenerative disease. Alternatively, the point mutation may comprise a G to A point mutation, and the deamination of the mutant A base by the fusion protein or RNP complex of the present disclosure may generate a sequence that is not associated with the retinal dysfunction, particularly retinal degenerative disease.

As used herein, the term “target site” refers to a sequence within a nucleic acid molecule that is deaminated by a deaminase or a fusion protein comprising a deaminase (for example, a dCas9-deaminase fusion protein provided herein). In an embodiment, the target sequence of the present disclosure comprises a point mutation associated with retinal dysfunction, such as retinal degenerative disease.

In another embodiment, the present disclosure provides a method comprising: contacting the target DNA molecule with (a) a Cas9 protein or fusion protein provided herein, and at least one gRNA, wherein the gRNA is about 15 to 100 nucleotides in length and contains a sequence of at least 10 contiguous nucleotides complementary to the target sequence; or (b) the RNP complex provided herein. In an embodiment, the contacting occurs in vivo in the subject.

In a specific embodiment, the present disclosure provides an in vivo gene correction method for treating retinal dysfunction, such as retinal degeneration, in a subject in need thereof, specifically, an in vivo gene correction method in a subject, comprising: delivering the fusion protein and gRNA; or the RNP complex to the subject together with Lipofectamine 2000. In this case, the fusion protein and gRNA; or the RNP complex may be delivered by a route suitable for treating retinal dysfunction, such as subretinal, subcutaneous, intradermal, intraocular, intravitreal route, and the like.

As used herein, the term “subject” refers to an individual organism, such as an individual mammal. In an embodiment, the subject is a human. In an embodiment, the subject is a non-human mammal, such as a sheep, goat, cow, cat, or dog.

In a specific embodiment, the RNP complex of the present disclosure may be used for in vivo gene correction in rd12 mice, specifically, correction of a nonsense mutation in the RPE65 gene that causes retinal degeneration.

As used herein, the term “retinal dysfunction” refers to any condition including any form, process and/or resulting outcome in which retinal tissue fails to perform its normal function or role.

As used herein, the term “retinal degeneration” refers to retinal deterioration caused by gradual and final death of retinal or retinal pigment epithelium (RPE) cells.

Retinal degeneration may be caused by several factors such as arterial or venous occlusion, diabetic retinopathy, retrolental fibroplasia/retinopathy of prematurity, or diseases (often genetic in nature). These may occur in several ways, such as visual impairment, night blindness, retinal detachment, photosensitivity, tunnel vision, and peripheral vision damage leading to total vision loss.

Retinal dysfunction of the present disclosure may include, but is not limited to, retinal degenerative disease; retinitis pigmentosa; retinitis pigmentosa; angiopathy; Drusen; lebers congenital amaurosis; hereditary or acquired macular degeneration; age-related macular degeneration (AMD); Best disease; retinal detachment; cerebral atrophy; choroidal defect; pattern dystrophy; retinal pigment epithelium (RPE) dystrophy; Stargardt disease; retinal pigment epithelium (RPE) and retinal damage caused by any one of light, laser, infection, radiation, neovascularization or traumatic injury; retinal dysplasia; color blindness; choroideremia; myopic choroidal neovascularization; nodular choroidal angiopathy; central serous chorioretinopathy; macular hole; macular dystrophy; diabetic retinopathy; retinal arteriovenous occlusion; hypertensive retinopathy; retinal aortic aneurysm; ophthalmic ischemia syndrome; retinopathy of prematurity; acute retinal necrosis; cytomegalovirus retinitis; toxoplasma retinochoroiditis; syphilitic chorioretinitis; retinal detachment; and retinoblastoma.

In particular, the retinal degenerative diseases of the present disclosure may include, but is not limited to, diseases listed above, such as retinal degenerative disease; retinitis pigmentosa; retinitis pigmentosa; angiopathy; Drusen; lebers congenital amaurosis; hereditary or acquired macular degeneration; age-related macular degeneration (AMD); Best disease; retinal detachment; cerebral atrophy; choroidal defect; pattern dystrophy; retinal pigment epithelium (RPE) dystrophy; Stargardt disease; retinal pigment epithelium (RPE) and retinal damage caused by any one of light, laser, infection, radiation, neovascularization or traumatic injury; and diseases related to this.

Retinal dysfunction, such as retinal degenerative disease, prevented or treated with the fusion protein or RNP complex of the present disclosure may include genetic mutations, especially point mutations. In an embodiment, the retinal dysfunction of the present disclosure, such as a retinal degenerative disease, may be associated with one or more gene mutations listed in Table 3. Thus, the gRNA of the present disclosure may target one or more of the following genes.

TABLE 3

| | | | | | |
|---|---|---|---|---|---|
| ABCA4 | PLA2G5 | C1QTNF5 | GABRB3 | NPHP3 | RIMS1 |
| ABHD12 | POMGNT1 | C2ORF71 | GALK1 | NPHP4 | RLBP1 |
| ADGRA3 | PRCD | CA4 | GALT | NPVF | ROM1 |
| ADIPOR1 | PRKCG | CABP4 | GCNT2 | NR2E3 | RP1 |
| AGBL5 | PROM1 | CACNA1F | GDF3 | NR2F1 | RP1L1 |
| AHI1 | PRPF3 | CACNA2D4 | GDF6 | NRL | RP2 |
| AHR | PRPF31 | CANT1 | GFER | NTF4 | RP9 |
| AIPL1 | PRPF4 | CAPN5 | GJA1 | NYX | RPE65 |
| ARHGEF18 | PRPF6 | CAV1 | GJA3 | OAT | RPGR |
| ARL2BP | PRPF8 | CBS | GJA8 | OCRL | RPGRIP1 |
| ARL3 | PRPH2 | CC2 D2A | GLI2 | OFD1 | RPGRIP1L |
| ARL6 | RBP3 | CDH23 | GNAT1 | OPA1 | RS1 |
| BBS1 | RBP4 | CDH3 | GNAT2 | OPA3 | RTN4IP1 |
| BBS10 | RD3 | CDHR1 | GNGT1 | OPN1LW | SAG |
| BBS12 | RDH11 | CEP290 | GNGT2 | OPN1MW | SALL2 |
| BBS2 | RDH12 | CERKL | GNPAT | OPN1SW | SBF2 |
| BBS4 | REEP6 | CFH | GPR98 | OPTC | SC5DL |
| BBS5 | RGR | CHD7 | GRIP1 | OPTN | SDCCAG8 |
| BBS7 | RHO | CHM | GRK1 | OTX2 | SEC23A |
| BBS9 | RLBP1 | CHMP4B | GRK7 | P3H2 | SEMA3E |
| BEST1 | ROM1 | CISD2 | GRM6 | PANK2 | SEMA4A |
| C1QTNF5 | RP1 | CJA8 | GUCA1A | PAX2 | SHH |
| C2ORF71 | RP1L1 | CLDN19 | GUCA1B | PAX6 | SIL1 |
| C8orf37 | RP2 | CLN3 | GUCA1C | PCDH15 | SIPA1L3 |
| CA4 | RP9 | CLPB | GUCY2D | PDC | SIX3 |
| CACNA1F | RPE65 | CLRN1 | GUCY2F | PDE6A | SIX5 |
| CC2D2A | RPGR | CLUL1 | HCCS | PDE6B | SIX6 |
| CDH23 | RPGRIP1 | CNGA1 | HESX1 | PDE6C | SLC16A12 |
| CDHR1 | RPGRIP1L | CNGA3 | HMCN1 | PDE6D | SLC1A7 |
| CEP290 | SAG | CNGB1 | HMGB3 | PDE6G | SLC24A1 |
| CERKL | SAMD11 | CNGB3 | HMX1 | PDE6H | SLC25A18 |
| CHM | SEMA4A | CNNM4 | HSF4 | PDZD7 | SLC25A22 |
| CLCC1 | SLC7A14 | COL11A1 | IDH3B | PEX1 | SLC25A46 |
| CLN3 | SNRNP200 | COL18A1 | IFT140 | PEX10 | SLC2A1 |
| CLRN1 | SPATA7 | COL2A1 | IGBP1 | PEX11B | SLC33A1 |
| CNGA1 | SPP2 | COL4A1 | IKBKG | PEX12 | SMOC1 |
| CNGB1 | TOPORS | COL 9A1 | IMPDH1 | PEX13 | SNRNP200 |
| CRB1 | TRIM32 | CRABP1 | IMPG2 | PEX14 | SNX3 |
| CRX | TRNT1 | CRABP2 | INPP5E | PEX16 | SOLH |
| CYP4V2 | TTC8 | CRB1 | INVS | PEX19 | SOX2 |
| DHDDS | TTPA | CRX | IQCB1 | PEX2 | SPATA7 |
| DHX38 | TUB | CRYAA | JAG1 | PEX26 | SPG7 |
| EMC1 | TULP1 | CRYAB | JAM3 | PEX3 | SRD5A3 |
| EYS | USH1C | CRYBA1 | KCNJ13 | PEX5L | SREBF2 |
| FAM161A | USH2A | CRYBA2 | KCNV2 | PEX6 | STRA6 |
| FLVCR1 | WFS1 | CRYBA4 | KIF11 | PEX7 | TBC1D20 |
| FSCN2 | WHRN | CRYBB1 | KLHL7 | PGK1 | TBK1 |
| GNAT1 | ZNF408 | CRYBB2 | LARGE | PHYH | TDRD7 |
| GNPTG | ZNF513 | CRYBB3 | LCA5 | PIGL | TEAD1 |
| GUCA1B | ABCA4 | CRYGB | LEMD2 | PITPNM3 | TEK |
| GUCY2D | ABCB6 | CRYGC | LEPREL1 | PITX2 | TENM3 |
| HGSNAT | ABCC6 | CRYGD | LIM2 | PITX3 | TFAP2A |
| HK1 | ABHD12 | CRYGS | LMX1B | POMGNT1 | TFPT |
| IDH3B | ACO2 | CTDP1 | LOXL1 | POMT1 | TGIF1 |
| IFT140 | ACTB | CTNNB1 | LRAT | POMT2 | TIMM8A |
| IFT172 | ACVR1 | CYP1B1 | LRIT1 | PORCN | TIMP3 |
| IMPDH1 | ADAM9 | CYP27A1 | LRP5 | PQBP1 | TLR4 |
| IMPG1 | ADAMTSL4 | CYP4V2 | LSS | PRCD | TMEM114 |
| IMPG2 | AFG3L2 | CYP51A1 | LTBP2 | PROM1 | TMEM126A |
| INPP5E | AGK | DFNB31 | m.11778G> | PRPF3 | TMEM67 |
| | | | A; | | |
| INVS | AGPS | DHCR7 | m. 14484T> | PRPF31 | TMEM70 |
| | | | C | | |
| IQCB1 | AHI1 | DHDDS | m.3460G>A; | PRPF6 | TMEM98 |
| KIAA1549 | AIPL1 | DMD | MAB21L2 | PRPF8 | TOPORS |
| KIZ | AKR1E2 | DNM1L | MAF | PRPH2 | TREX1 |
| KLHL7 | ALDH18A1 | EFEMP1 | MAN2B1 | PRSS56 | TRIM32 |
| LCA5 | ALDH1A1 | ELOVL4 | MERTK | PVRL3 | TRPM1 |
| LRAT | ALDH1A2 | EPG5 | MEN2 | PXDN | TSPAN12 |
| MAK | ALDH1A3 | EPHA2 | MFRP | RAB18 | TTC8 |
| MERTK | ALMS1 | ERCC1 | MFSD6L | RAB3GAP1 | TTPA |
| MFRP | ANTXR1 | ERCC2 | MIP | RAB3GAP2 | TTR |
| MKKS | AOC2 | ERCC3 | MIR184 | RARA | TULP1 |
| MTATP6 | ARL6 | ERCC5 | MITF | RARB | UNC119 |
| MTTS2 | ARR3 | ERCC6 | MKKS | RARG | UNC45B |
| MVK | ASB10 | ERCC8 | MKS1 | RAX | USH1C |
| NEK2 | ATOH7 | EYA1 | MPP4 | RAX2 | USH1G |
| NEUROD1 | ATXN7 | EYS | MSMO1 | RB1 | USH2A |
| NMNAT1 | B3GALTL | FAM126A | MT-ATP6 | RBP1 | VAX1 |
| NPHP1 | B3GLCT | FAM161A | MT-CO1 | RBP3 | VCAN |
| NPHP3 | BBS1 | FBLN5 | MT-CO3 | RBP4 | VIM |
| NPHP4 | BBS10 | FBN1 | MTCYB | RCBTB1 | VPS13B |
| NR2E3 | BBS12 | FDXR | MT-ND1 | RCVRN | VSX1 |
| NRL | BBS2 | FKRP | MT-ND2 | RD3 | VSX2 |
| OFD1 | BBS4 | FKTN | MT-ND4 | RDH10 | WDR36 |
| PANK2 | BBS5 | FLVCR1 | MT-ND5 | RDH11 | WFS1 |
| PCARE | BBS7 | FOXC1 | MT-ND6 | RDH12 | WR |
| PCDH15 | BBS9 | FOXD3 | MTTP | RDH5 | YAP1 |
| PDE6A | BCMO1 | FOXE3 | MYO7A | RDH8 | YME1L1 |
| PDE6B | BCOR | FOXL2 | MYOC | RECQL2 | ZIC2 |
| PDE6G | BEST1 | FRAS1 | NAA10 | RECQL4 | ZNF408 |
| PEX1 | BESP1 | FREM1 | NCALD | RGR | ZNF513 |
| PEX2 | BFSP2 | FREM2 | NDP | RGS11 | |
| PEX26 | BMP4 | FSCN2 | NF2 | RGS16 | |
| PEX7 | BMP7 | FTL | NHS | RGS9 | |
| PHYH | C12orf57 | FYCO1 | NMNAT1 | RGS9BP | |
| PITPNM3 | C12ORF65 | FZD4 | NPHP1 | RHO | |

In an embodiment, the pharmaceutical composition of the present disclosure may comprise an effective amount of the fusion protein or RNP complex. In another embodiment, the gene editing method of the present disclosure may be used to deliver an effective amount of the fusion protein or RNP complex to a subject.

As used herein, the term “effective amount” refers to an amount of biologically active agent sufficient to cause a desired biological response. For example, in an embodiment, the effective amount of the fusion protein or RNP complex may refer to an amount sufficient to induce editing of the target site specifically bound and edited by the fusion protein or RNP complex.

In another embodiment, the present disclosure provides a method for preventing or treating retinal dysfunction comprising administering the fusion protein or RNP complex to a subject in need thereof.

In still another embodiment, the present disclosure provides uses of the fusion protein or RNP complex for the prevention or treatment of retinal dysfunction.

In still another embodiment, the present disclosure provides uses of the fusion protein or RNP complex for use in the manufacture of a medicament for use in the prevention or treatment of retinal dysfunction.

The fusion protein, RNP complex, and retinal dysfunction are the same as described above.

### Purification Method of Fusion Protein

The present disclosure provides a method for purifying a fusion protein with high purity.

In an embodiment, the present disclosure provides a purification method of a fusion protein, comprising:
(a) expressing a fusion protein in a cell, the fusion protein containing a Cas9 domain; and an adenine deaminase domain or a cytidine deaminase domain, wherein the fusion protein further includes an affinity tag;
(b) dissolving the fusion protein expressed in Step (a) to produce a lysate;
(c) performing primary purification on the lysate of Step (b) by affinity chromatography;
(d) performing secondary purification on the fusion protein purified in Step (c) by affinity chromatography; and
(e) performing third purification on the fusion protein purified in Step (d) by size exclusion chromatography.

In a specific embodiment, the affinity tag may be a polyhistidine tag or a FLAG tag, for example, the fusion protein of Step (a) may comprise a 10×His-Flag tag at the N-terminus and an mCherry-10×His tag at the C-terminus.

In a specific embodiment, Step (c) may comprise contacting the lysate of Step (b) with a Ni-NTA resin, wherein the fusion protein may be bound to the Ni-NTA resin.

In a specific embodiment, Step (d) may comprise contacting the fusion protein purified in Step (c) with an α-FLAG M1 agarose resin, wherein the fusion protein may be bound to the α-FLAG M1 agarose resin.

In a specific embodiment, the size exclusion chromatography of Step (e) may be performed on a HiLoad 16/600 Superdex 200 pg, HiLoad 26/600 Superdex 200 pg, or HiLoad 16/600 Superdex 75 pg column, and specifically, the HiLoad 16/600 Superdex 200 pg column may be used.

Further, the present disclosure provides a purification method of a fusion protein, comprising:
(a) expressing a fusion protein in a cell, the fusion protein containing a Cas9 domain; and an adenine deaminase domain or a cytidine deaminase domain, wherein the fusion protein further includes a 10×His-Flag tag at the N-terminus and an mCherry—at the C-terminus;
(b) dissolving the fusion protein expressed in Step (a) to produce a lysate;
(c) performing primary purification on the lysate of Step (b) by affinity chromatography, which comprises contacting the lysate with a Ni-NTA resin, wherein the fusion protein is bound to the Ni-NTA resin;
(d) performing secondary purification by affinity chromatography by contacting the fusion protein purified in Step (c) with the Ni-NTA resin, which comprises contacting the fusion protein purified in Step (c) with an α-FLAG M1 agarose resin, wherein the fusion protein is bound to the α-FLAG M1 agarose resin; and
(e) performing third purification on the fusion protein purified in Step (d) by size exclusion chromatography, wherein the size exclusion chromatography is performed on a HiLoad 16/600 Superdex 200 pg, HiLoad 26/600 Superdex 200 pg, or HiLoad 16/600 Superdex 75 pg column.

In an embodiment, the present disclosure provides a fusion protein purified according to the above purification method.

In still another embodiment, the present disclosure provides a ribonucleoprotein (RNP) complex comprising: a fusion protein purified according to the above purification method; and a guide RNA (gRNA) bound to the Cas9 domain of the fusion protein.

Hereinafter, the present disclosure will be described in more detail through Experimental methods and Examples. These Examples are only provided for illustrating the present disclosure in more detail, and it will be apparent to those skilled in the art that the scope of the present disclosure is not limited by these Examples according to the gist of the present disclosure.

### Experimental Method

The present disclosure may be carried out by the following Experimental method.

### 1. Preparation of Plasmid Encoding Base Editor

To prepare pEX-FlagR-ABEmax and pEX-FlagR-BE4max, the ABEmax and BE4max coding sequences (CDS) were amplified from pCMV_ABEmax (addgene no. 112095) and pCMV_AncBE4max (addgene no. 112094), respectively, and cloned into the mammalian expression pEX-FlagR vector using XhoI and XbaI restriction sites.

To construct pCMV-NGABEmax, the Cas9 sequence of pCMV_ABEmax was replaced with the SpCas9-NG sequence of pX330-SpCas9-NG (addgene no. 117919) using PmlI and EcoRI restriction sites.

To construct pEX-FlagR-NGABEmax, the NGABEmax sequence of pCMV-NGABEmax was cloned into pEX-FlagR. Gibson fragments containing the ABEmax or BE4max CDS with matching overlap were PCR amplified using Phusion High-Fidelity Polymerase (NEB). Fragments were gel purified, assembled using NEBuilder HiFi DNA Assembly master mix (NEB) for 1 hour at 50° C., and transformed into chemical-compatible E. coli (DH5a, Enzynomics). The sequence corresponding to the sgRNA was cloned into the BsaI-digested pRG2 vector (Addgene no. 104174). For this step, oligos containing spacer sequences (Table 4) were annealed to form double-stranded DNA fragments with compatible overhangs and subsequently ligated using T4 ligase (Enzynomics). All plasmids used in the transfection experiments were employed for the NucleoBond Xtra Midi Plus EF kit (MN).

Table 4 below shows the primers used to prepare gRNAs. The 20-nt target protospacers are underlined. When the target DNA sequence does not start with ‘G’, since the human U6 promoter prefers ‘G’ at the transcription start site, ‘G’ was added at the 5′ end of the primer (gX20 otherwise GX19)

TABLE 4

| Primer | Sequences | SEQ NO. |
|---|---|---|
| F-ABE_site 2 | 5′-CACCGAGTATGAGGCATAGACTGC-3′ | 1 |
| R-ABE_site 2 | 5′-AAACGCAGTCTATGCCTCATACTC-3′ | 2 |
| F-ABE_site 4 | 5′-CACCGAGCAAAGAGAATAGACTGT-3′ | 3 |
| R-ABE_site 4 | 5′-AAACACAGTCTATTCTCTTTGCTC-3′ | 4 |
| F-ABE_site 5 | 5′-CACCGATGAGATAATGATGAGTCA-3′ | 5 |
| R-ABE_site 5 | 5′-AAACTGACTCATCATTATCTCATC-3′ | 6 |
| F-ABE_site 8 | 5′-CACCGTAAACAAAGCATAGACTGA-3′ | 7 |
| R-ABE_site 8 | 5′-AAACTCAGTCTATGCTTTGTTTAC-3′ | 8 |
| F-ABE_site 10 | 5′-CACCGAACATAAAGAATAGAATGA-3′ | 9 |
| R-ABE_site 10 | 5′-AAACTCATTCTATTCTTTATGTTC-3′ | 10 |
| F-ABE_site 13 | 5′-CACCGAAGATAGAGAATAGACTGC-3′ | 11 |
| R-ABE_site 13 | 5′-AAACGCAGTCTATTCTCTATCTTC-3′ | 12 |
| F-ABE_site 16 | 5′-CACCGGGAATAAATCATAGAATCC-3′ | 13 |
| R-ABE_site 16 | 5′-AAACGGATTCTATGATTTATTCCC-3′ | 14 |
| F-ABE_site 17 | 5′-CACCGACAAAGAGGAAGAGAGACG-3′ | 15 |
| R-ABE_site 17 | 5′-AAACCGTCTCTCTTCCTCTTTGTC-3′ | 16 |
| F-ABE_site 28 | 5′-CACCGACAAACCAGAAGCCGCTCC-3′ | 17 |
| R-ABE_site 28 | 5′-AAACGGAGCGGCTTCTGGTTTGTC-3′ | 18 |
| F-CCR5_site 10 | 5′-CACCGGTGACAAGTGTGATCACTT-3′ | 19 |
| R-CCR5_site 10 | 5′-AAACAAGTGATCACACTTGTCACC-3′ | 20 |
| F-FANCE | 5′-CACCGGAATCCCTTCTGCAGCACC-3′ | 21 |
| R-FANCF | 5′-AAACGGTGCTGCAGAAGGGATTCC-3′ | 22 |
| F-HBG_site 1 | 5′-CACCGCTTGACCAATAGCCTTGACA-3′ | 23 |
| R-HBG_site 1 | 5′-AAACTGTCAAGGCTATTGGTCAAGC-3′ | 24 |
| F-HBG_site 2 | 5′-CACCGATATTTGCATTGAGATAGTG-3′ | 25 |
| R-HBG_site 2 | 5′-AAACCACTATCTCAATGCAAATATC-3′ | 26 |
| F-HBG_site 3 | 5′-CACCGTGGGGAAGGGGCCCCCAAG-3′ | 27 |
| R-HBG_site 3 | 5′-AAACCTTGGGGGCCCCTTCCCCAC-3′ | 28 |
| F-HEK293_site 2 | 5′-CACCGAACACAAAGCATAGACTGC-3′ | 29 |
| R-HEK293_site 2 | 5′-AAACGCAGTCTATGCTTTGTGTTC-3′ | 30 |
| F-HEK293_site 3 | 5′-CACCGGCCCAGACTGAGCACGTGA-3′ | 31 |
| R-HEK293_site 3 | 5′-AAACTCACGTGCTCAGTCTGGGCC-3′ | 32 |
| F-HEK293_site 4 | 5′-CACCGGCACTGCGGCTGGAGGTCC-3′ | 33 |
| R-HEK293_site 4 | 5′-AAACGGACCTCCAGCCGCAGTGCC-3′ | 34 |
| F-HPRT Exon 4 | 5′-CACCGGGGACATAAAAGTAATTGG-3′ | 35 |
| R-HPRT Exon 4 | 5′-AAACCCAATTACTTTTATGTCCCC-3′ | 36 |
| F-HPRT Exon 8 | 5′-CACCGAAGTATTCATTATAGTCAA-3′ | 37 |
| R-HPRT Exon 8 | 5′-AAACTTGACTATAATGAATACTTC-3′ | 38 |
| F-VEGFA | 5′-CACCGGTGAGTGAGTGTGTGCGTG-3′ | 39 |
| R-VEGFA | 5′-AAACCACGCACACACTCACTCACC-3′ | 40 |
| F-PPP1R12C_site 3 | 5′-CACCGACCCTCAGCCGTGCTGCTC-3′ | 41 |
| R-PPP1R12C_site 3 | 5′-AAACGAGCAGCACGGCTGAGGGTC-3′ | 42 |
| F-PPP1R12C_site 4 | 5′-CACCGCTCTCAGCCTGGAGACCAC-3′ | 43 |
| R-PPP1R12C_site 4 | 5′-AAACGTGGTCTCCAGGCTGAGAGC-3′ | 44 |
| F-PPP1R12C_site 6 | 5′-CACCGGGGCTCAACATCGGAAGAG-3′ | 45 |
| R-PPP1R12C_site 6 | 5′-AAACCTCTTCCGATGTTGAGCCCC-3′ | 46 |
| F-PPP1R12C_site 9 | 5′-CACCGCTGGCTCAGGTTCAGGAGA-3′ | 47 |
| R-PPP1R12C_site 9 | 5′-AAACTCTCCTGAACCTGAGCCAGC-3′ | 48 |
| F-EMX1 | 5′-CACCGAGTCCGAGCAGAAGAAGAA-3′ | 49 |
| R-EMX1 | 5′-AAACTTCTTCTTCTGCTCGGACTC-3′ | 50 |
| F-EMX1_GGT PAM | 5′-CACCGCAGGGATCCAAGCACACAA-3′ | 51 |
| R-EMX1_GGT PAM | 5′-AAACTTGTGTGCTTGGATCCCTGC-3′ | 52 |
| F-VEGFA_CGT PAM | 5′-CACCGCTCTCAGGCCCTGTCCGCA-3′ | 53 |
| R-VEGFA_CGT PAM | 5′-AAACTGCGGACAGGGCCTGAGAGC-3′ | 54 |
| F-GRIN2B_AGA PAM | 5′-CACCGAGCAAATACCAGAGATAAG-3′ | 55 |
| R-GRIN2B_AGA PAM | 5′-AAACCTTATCTCTGGTATTTGCTC-3′ | 56 |
| F-FANCF_GGA PAM | 5′-CACCGAGAACCCAAATCTCCAGGA-3′ | 57 |
| R-FANCF_GGA PAM | 5′-AAACTCCTGGAGATTTGGGTTCTC-3′ | 58 |
| F-PTEN_GGA PAM | 5′-CACCGTACCTAATGGACTTCAGGG-3′ | 59 |
| R-PTEN_GGA PAM | 5′-AAACCCCTGAAGTCCATTAGGTAC-3′ | 60 |
| F-MECP2_AGT PAM | 5′-CACCGCCTTCATAGAATTGAAGAG-3′ | 61 |
| R-MECP2_AGT PAM | 5′-AAACCTCTTCAATTCTATGAAGGC-3′ | 62 |

### 2. Fusion Protein Expression and Purification

Human embryonic kidney 293 EBNA1 (HEK293E) cells were grown at 37° C. in suspension in Dulbecco's Modified Eagle Medium (DMEM) with 4500 mg/L calcium-free glucose (WELGENE) supplemented with 5% fetal bovine serum (FBS). For overexpression of base editors (ABEmax or BE4max), HEK293E cells were transiently transfected with pEX-FlagR-ABEmax (NG PAM or NGG PAM) or pEX-FlagR-AncBE4max (NGG PAM) plasmids, which was designed so that each base editor was expressed as a fusion protein with a 10×His-Flag tag at the N terminus and a mCherry-10×His tag at the C terminus. Cells were transfected with 25 kDa linear polyethyleneimine (Polysciences) at a density of 7×10⁵ cells/mL. Immediately after transfection, dimethyl sulfoxide (Amresco) was added to a final concentration of 1% and the temperature was lowered to 33° C. Two days after transfection, tryptone (Amresco) was added to a final concentration of 0.5%. Four days after transfection, cells were harvested at 500 g for 20 minutes and resuspended in lysis buffer [20 mM Tris-HCl (pH 7.5), 1 M NaCl, and 2 mM β-mercaptoethanol]supplemented with 20% glycerol. The resuspended cells were lysed by sonication. The resulting solution was centrifuged and the supernatant was loaded onto a Ni-NTA column (Qiagen). The column was washed with buffer A [20 mM Tris-HCl (pH 7.5), 500 mM NaCl, 2 mM β-mercaptoethanol and 20% glycerol](MilliporeSigma) supplemented with 40 mM imidazole, and the bound proteins were then eluted with buffer A supplemented with 200 mM imidazole. The eluted proteins were treated overnight with tobacco etch virus (TEV) protease and human rhinovirus (HRV) 3C protease to expose the FLAG tag at the N terminus and remove the C terminal mCherry-10×His tag, respectively. Samples were mixed with α-FLAG M1 agarose resin (MilliporeSigma) in the presence of 5 mM CaCl₂) and agitated gently for 1 hour at 4° C. The samples were washed with buffer A supplemented with 1 mM CaCl₂), and the bound proteins were then eluted with buffer A supplemented with 5 mM EGTA. The eluted FLAG-ABEmax (or FLAG-CBEmax) was concentrated and further purified using a HiLoad 16/600 Superdex 200 pg column equilibrated with a buffer containing 20 mM Tris-HCl (pH 7.5), 500 mM NaCl, 1 mM β-mercaptoethanol, and 20% glycerol (storage buffer). The peak fractions were then concentrated to about 10 mg/mL, rapidly frozen in liquid nitrogen, and stored at −80° C.

3. Preparation of Guide RNA (sgRNA)

For RNP delivery of the base editor, sgRNA was synthesized by in vitro transcription using T7 RNA polymerase and template oligonucleotide (Table 5). In vitro transcribed sgRNA targeting the rd12 allele was further treated with calf intestinal alkaline phosphatase (CIP, NEB) to remove 5′-triphosphate (gX19+CIP). A chemically synthesized sgRNA targeting the rd12 allele (X19 IDT and X20_IDT) containing chemical modifications (Alt-R sgRNA) was purchased from Integrated DNA Technologies (IDT), Inc.

Table 5 below shows the primers used to generate in vitro transcribed gRNAs. The 20-nt target protospacers are underlined.

TABLE 5

| Primer | Sequences | SEQ NO. |
|---|---|---|
| R-sgRNA | 5′-AAAAAAAGCACCGACTCGGTGCCACTTTTTCAAGTTG | 63 |
| scaffold | ATAACGGACTAGCCTTATTTAAACTTGCTATGCTGTTTCC | |
| | AGCATAGCTCTTAAAC-3′ | |
| F-ABE_site 2 | 5′-GAAATTAATACGACTCACTATAGAGTATGAGGCATAG | 64 |
| | ACTGCGTTTAAGAGCTATGCTGGAAAC-3′ | |
| F-ABE_site 4 | 5′-GAAATTAATACGACTCACTATAGAGCAAAGAGAATAG | 65 |
| | ACTGTGTTTAAGAGCTATGCTGGAAAC-3′ | |
| F-ABE_site 5 | 5′-GAAATTAATACGACTCACTATAGATGAGATAATGATG | 66 |
| | AGTCAGTTTAAGAGCTATGCTGGAAAC-3′ | |
| F-ABE_site 8 | 5′-GAAATTAATACGACTCACTATAGTAAACAAAGCATAG | 67 |
| | ACTGAGTTTAAGAGCTATGCTGGAAAC-3′ | |
| F-ABE_site 10 | 5′-GAAATTAATACGACTCACTATAGAACATAAAGAATAG | 68 |
| | AATGAGTTTAAGAGCTATGCTGGAAAC-3′ | |
| F-ABE_site 13 | 5′-GAAATTAATACGACTCACTATAGAAGATAGAGAATAG | 69 |
| | ACTGCGTTTAAGAGCTATGCTGGAAAC-3′ | |
| F-ABE_site 16 | 5′-GAAATTAATACGACTCACTATAGGGAATAAATCATAG | 70 |
| | AATCCGTTTAAGAGCTATGCTGGAAAC-3′ | |
| F-ABE_site 17 | 5′-GAAATTAATACGACTCACTATAGACAAAGAGGAAGAG | 71 |
| | AGACGGTTTAAGAGCTATGCTGGAAAC-3′ | |
| F-ABE_site 28 | 5′-GAAATTAATACGACTCACTATAGACAAACCAGAAGCC | 72 |
| | GCTCCGTTTAAGAGCTATGCTGGAAAC-3′ | |
| F-CCR5_site 10 | 5′-GAAATTAATACGACTCACTATAGGTGACAAGTGTGAT | 73 |
| | CACTTGTTTAAGAGCTATGCTGGAAAC-3′ | |
| F-FANCF | 5′-GAAATTAATACGACTCACTATAGGAATCCCTTCTGCA | 74 |
| | GCACCGTTTAAGAGCTATGCTGGAAAC-3′ | |
| F-HBG_site 1 | 5′-GAAATTAATACGACTCACTATAGCTTGACCAATAGCC | 75 |
| | TTGACAGTTTAAGAGCTATGCTGGAAAC-3′ | |
| F-HBG_site 2 | 5′-GAAATTAATACGACTCACTATAGATATTTGCATTGAG | 76 |
| | ATAGTGGTTTAAGAGCTATGCTGGAAAC-3′ | |
| F-HBG_site 3 | 5′-GAAATTAATACGACTCACTATAGTGGGGAAGGGGCCC | 77 |
| | CCAAGGTTTAAGAGCTATGCTGGAAAC-3′ | |
| F-HEK293_site 2 | 5′-GAAATTAATACGACTCACTATAGAACACAAAGCATAG | 78 |
| | ACTGCGTTTAAGAGCTATGCTGGAAAC-3′ | |
| F-HEK293_site 3 | 5′-GAAATTAATACGACTCACTATAGGCCCAGACTGAGCA | 79 |
| | CGTGAGTTTAAGAGCTATGCTGGAAAC-3′ | |
| F-HEK293_site 4 | 5′-GAAATTAATACGACTCACTATAGGCACTGCGGCTGGA | 80 |
| | GGTCCGTTTAAGAGCTATGCTGGAAAC-3′ | |
| F-HPRT Exon 4 | 5′-GAAATTAATACGACTCACTATAGGGGACATAAAAGTA | 81 |
| | ATTGGGTTTAAGAGCTATGCTGGAAAC-3′ | |
| F-HPRT Exon 8 | 5′-GAAATTAATACGACTCACTATAGAAGTATTCATTATA | 82 |
| | GTCAAGTTTAAGAGCTATGCTGGAAAC-3′ | |
| F-VEGFA | 5′-GAAATTAATACGACTCACTATAGGTGAGTGAGTGTGT | 83 |
| | GCGTGGTTTAAGAGCTATGCTGGAAAC-3′ | |
| F- | 5′-GAAATTAATACGACTCACTATAGACCCTCAGCCGTGC | 84 |
| PPP1R12C_site 3 | TGCTCGTTTAAGAGCTATGCTGGAAAC-3′ | |
| F- | 5′-GAAATTAATACGACTCACTATAGCTCTCAGCCTGGAG | 85 |
| PPP1R12C_site 4 | ACCACGTTTAAGAGCTATGCTGGAAAC-3′ | |
| F- | 5′-GAAATTAATACGACTCACTATAGGGGCTCAACATCGG | 86 |
| PPP1R12C_site 6 | AAGAGGTTTAAGAGCTATGCTGGAAAC-3′ | |
| F- | 5′-GAAATTAATACGACTCACTATAGCTGGCTCAGGTTCA | 87 |
| PPP1R12C_site 9 | GGAGAGTTTAAGAGCTATGCTGGAAAC-3′ | |
| F-EMX1 | 5′-GAAATTAATACGACTCACTATAGAGTCCGAGCAGAAG | 88 |
| | AAGAAGTTTAAGAGCTATGCTGGAAAC-3′ | |
| F-EMX1_GGT PAM | 5′-GAAATTAATACGACTCACTATAGCAGGGATCCAAGCA | 89 |
| | CACAAGTTTAAGAGCTATGCTGGAAAC-3′ | |
| F-VEGFA_CGT PAM | 5′-GAAATTAATACGACTCACTATAGCTCTCAGGCCCTGT | 90 |
| | CCGCAGTTTAAGAGCTATGCTGGAAAC-3′ | |
| F-GRIN2B_AGA PAM | 5′-GAAATTAATACGACTCACTATAGAGCAAATACCAGAG | 91 |
| | ATAAGGTTTAAGAGCTATGCTGGAAAC-3′ | |
| F-FANCF_GGA PAM | 5′-GAAATTAATACGACTCACTATAGAGAACCCAAATCTC | 92 |
| | CAGGAGTTTAAGAGCTATGCTGGAAAC-3′ | |
| F-PTEN_GGA PAM | 5′-GAAATTAATACGACTCACTATAGTACCTAATGGACTT | 93 |
| | CAGGGGTTTAAGAGCTATGCTGGAAAC-3′ | |
| F-MECP2_AGT PAM | 5′-GAAATTAATACGACTCACTATAGCCTTCATAGAATTG | 94 |
| | AAGAGGTTTAAGAGCTATGCTGGAAAC-3′ | |
| F-Target rd12 | 5′-GAAATTAATACGACTCACTATAGACATCAGAGGAGAC | 95 |
| gx20 | TGCCAGGTTTAAGAGCTATGCTGGAAAC-3′ | |
| F-Target rd12 | 5′-GAAATTAATACGACTCACTATAGCATCAGAGGAGACT | 96 |
| gx19 | GCCAGGTTTAAGAGCTATGCTGGAAAC-3′ | |

4. Preparation of ABE-Encoding mRNA

pCMV_ABEmax was linearized by PmeI digestion and used as a template for in vitro synthesis of ABE encoding mRNA. The ABE encoding mRNA was transcribed using the mMESSAGE mMACHINE T7 Transcription kit (Invitrogen), producing mRNA co-transcriptionally capped with 7-methylguanosine at the 5′ end. The 3′ end of the mRNA was then polyadenylated using the Poly(A) Tailing Kit (Invitrogen) according to the manufacturer's instructions.

### 5. Cell Culture and Transfection for Base-Editing Experiments

HEK293T cells (ATCC CRL-11268) were cultured in DMEM (WELGENE) supplemented with 10% FBS and 1% penicillin-streptomycin.

For ABE or CBE RNP-mediated genome editing, 15 μg of base editor protein (10 mg/ml of ABE or 8 mg/ml of CBE dissolved in storage buffer) and 8 μg of in vitro transcribed or chemically synthesized sgRNAs were mixed and incubated for 10 minutes at room temperature to form an RNP complex. The RNP complex was then mixed with HEK293T cells (1.5×10⁵) and electroporated through the Neon Transfection System.

For ABE RNP-mediated RNA off-target editing and time course analysis, HEK293T cells (1.5×10⁵) were electroporated as above without sgRNA.

For ABE or CBE encoding plasmid-mediated genome editing, ABE or CBE encoding plasmid (0.5 μg) and sgRNA encoding plasmid (0.17 μg) were mixed with HEK293T cells (1.5×10⁵) and electroporated through the Neon Transfection System.
For ABE encoding mRNA-mediated genome editing, ABE encoding mRNA (3.0 μg) and various doses of sgRNA (0.6, 1.5, 3.0, 8.0 or 16.0 μg) were mixed with HEK293T cells (1.5×10⁵) and electroporated with the Neon Transfection System.
For orthogonal R-loop analysis using the base editor plasmid, 500 ng of dead SaCas9 plasmid (addgene no. 138162), 170 ng of SaCas9 sgRNA plasmid, 500 ng of base editor plasmid, and 170 ng of base editor sgRNA plasmid were co-transfected into HEK293T cells (1.0×10⁵) using 2 μL of Lipofectamine 2000 (cat no. 11668019, Thermo).
For orthogonal R-loop analysis using base editor RNP, 500 ng of dead SaCas9 plasmid, 170 ng of SaCas9 sgRNA plasmid, and 670 ng of pUC19 plasmid (negative control plasmid) were co-transfected into HEK293T cells (1.0×10⁵) using 2 μL of Lipofectamine 2000.
One day after transfection, cells treated without base editor plasmid were treated with trypsin and centrifuged at 100×g for 8 minutes. The cells were resuspended, and electroporated with base editor protein (15 μg) and in vitro transcribed sgRNA (8 μg) via the Neon Transfection System.
For in vitro rd12 mutation correction, mEFs from rd12 mice were maintained in DMEM supplemented with 10% FBS, 1% penicillin-streptomycin (WELGENE), and 4 mM glutamine (Glutamax-I, Gibco). For ABE-RNP-mediated gene editing, mEFs (1.5×10⁵) were electroporated with NGABEmax protein (15 μg) and in vitro transcribed sgRNA (8 μg), CIP-treated in vitro transcribed sgRNA (8 μg), or chemically synthesized sgRNA (IDT, 8 μg) through the Neon Transfection System.

### 6. Evaluation of ABE Expression Level Through Western Blot Analysis

Cell lysates were prepared from ABE-transfected HEK293T cells using RIPA buffer (SIGMA) supplemented with protease inhibitor cocktail (SIGMA). Protein concentration was measured using the BCA assay kit (Thermo Fisher). The same amounts of protein were loaded onto Mini Protean TGX Protein Gel (BioRad) and run at 80 V for 20 minutes and at 120 V for minutes. The proteins were transferred to a nitrocellulose membrane, and the blots were incubated with anti-Cas9 (#844301, BioLegend) and anti-tubulin (#3873, Cell Signaling) antibodies, followed by incubation with appropriate horseradish peroxidase (HRP) conjugated secondary antibody (#7076, Cell Signaling). Chemiluminescence of the HRP reaction was detected using the Fusion SL gel chemiluminescence documentation system (Vilber Lourmat).

### 7. Animals Used in Experiments

Mated pairs of 8-week-old male C57BL/6 mice and rd12 mice (stock no. 005379, The Jackson Laboratory) were purchased through Central Laboratory Animal and maintained under a 12-hour dark-light cycle. All animal experiments were conducted in accordance with the guidelines of the Association for Research in Vision and Ophthalmology Statement on the Use of Animals in Ophthalmic and Vision Research.

### 8. Mouse Subretinal Injection

3-Week-old mice (juvenile mice) or 6-month-old mice (adult mice) were anesthetized. Mice were injected subretinally with a 1:1 (v/v) mixture of RNP and Lipofectamine® 2000 (cat no. 11668019, Thermo) in one eye using a custom Nanofil syringe (World Precision Instrument) equipped with a 33 gauge blunt needle. Each dose contained 12.54 μg NG-ABEmax and 5.76 μg of appropriate sgRNA. The total volume per eye was 3 μL.

### 9. Targeted Deep Sequencing—On-Target and Off-Target Editing Analysis

For analysis of DNA or RNA on-target and off-target sites, genomic DNA or total RNA was extracted from ABE-transfected cells using the NucleoSpin Tissue Kit (MN) or NucleoSpin RNA Plus Kit (MN) at indicated time points or 3 days after transfection. cDNA was synthesized from RNA using PrimeScript RT master mix (TAKARA). For sequencing of ABE target regions in normal or rd12 mice, the genome DNAs were extracted from retinal pigment epithelial (RPE) cells using the NucleoSpin Tissue Kit (MN) at 1 or 5 weeks after subretinal injection of the ABE RNP/Lipofectamine 2000 mixture. To generate a sequence library, on-target and off-target sites were amplified using the KOD Multi & Epi PCR kit (TOYOBO) (Tables 6 and 7). These libraries were sequenced using MiniSeq (Illumina) with the TruSeq HT Dual Index system. In other words, the same amount of PCR amplicons was subjected to paired-end read sequence analysis using the Illumina MiniSeq platform. After MiniSeq, paired-end reads were analyzed by comparing wild type and mutant sequences using BE-analyzer.

Table 6 below shows the primers used for analysis of DNA on-target editing. The 5′ tail sequences for extension of the HT True seq index are underlined.

TABLE 6

| Primer | Sequences | SEQ NO. |
|---|---|---|
| fwd-ABE_site 2 | ACACTCTTTCCCTACACGACGCTCTTCCGATC | 97 |
| | TGGGAACCTCAGGTGAAAAGTC | |
| rev-ABE_site 2 | GTGACTGGAGTTCAGACGTGTGCTCTTCCGAT | 98 |
| | CTCCTAGGCAACAAGAGCAAAA | |
| fwd-ABE_site 4 | ACACTCTTTCCCTACACGACGCTCTTCCGATC | 99 |
| | TTCTCGATCTCCTGACCTCGT | |
| rev-ABE_site 4 | GTGACTGGAGTTCAGACGTGTGCTCTTCCGAT | 100 |
| | CTTGTCAGGTAATGTGCTAAACAGAGA | |
| fwd-ABE_site 5 | ACACTCTTTCCCTACACGACGCTCTTCCGATC | 101 |
| | TAAGTCAAGCCTGAGCTTCCA | |
| rev-ABE_site 5 | GTGACTGGAGTTCAGACGTGTGCTCTTCCGAT | 102 |
| | CTCCCAAAGTGCTGGGATTTTA | |
| fwd-ABE_site 8 | ACACTCTTTCCCTACACGACGCTCTTCCGATC | 103 |
| | TGCCGTGGGAGACAATTCATA | |
| rev-ABE_site 8 | GTGACTGGAGTTCAGACGTGTGCTCTTCCGAT | 104 |
| | CTGGTTCTGTTTGTGGCCAAG | |
| fwd-ABE_site 10 | ACACTCTTTCCCTACACGACGCTCTTCCGATC | 105 |
| | TAAAGTCACTCTGCTGTGCTATTAGA | |
| rev-ABE_site 10 | GTGACTGGAGTTCAGACGTGTGCTCTTCCGAT | 106 |
| | CTTGAGCAAGGCACAGAAAGAC | |
| fwd-ABE_site 13 | ACACTCTTTCCCTACACGACGCTCTTCCGATC | 107 |
| | TTCAAACACTGGGTCATACTTCTTC | |
| rev-ABE_site 13 | GTGACTGGAGTTCAGACGTGTGCTCTTCCGAT | 108 |
| | CTCCATCTGGGCACAGTGTTAC | |
| fwd-ABE_site 16 | ACACTCTTTCCCTACACGACGCTCTTCCGATC | 109 |
| | TCCACCTGGAATGAGTTTTCG | |
| rev-ABE_site 16 | GTGACTGGAGTTCAGACGTGTGCTCTTCCGAT | 110 |
| | CTATGCCAGATACCAGCAATCC | |
| fwd-ABE_site 17 | ACACTCTTTCCCTACACGACGCTCTTCCGATC | 111 |
| | TCAAGCCTGATTCCAAGGAGA | |
| rev-ABE_site 17 | GTGACTGGAGTTCAGACGTGTGCTCTTCCGAT | 112 |
| | CTAAATGAGCCTCTGGTGGAGA | |
| fwd-ABE_site 28 | ACACTCTTTCCCTACACGACGCTCTTCCGATC | 113 |
| | TTTCTTGTAGCCCTCTTTTTATTGG | |
| rev-ABE_site 28 | GTGACTGGAGTTCAGACGTGTGCTCTTCCGAT | 114 |
| | CTCCTATCACAATCACAACTGCAA | |
| fwd-CCR5_site 10 | ACACTCTTTCCCTACACGACGCTCTTCCGATC | 115 |
| | TCCTGACAATCGATAGGTACC | |
| rev-CCR5_site 10 | GTGACTGGAGTTCAGACGTGTGCTCTTCCGAT | 116 |
| | CTAGCCCCAAGATGACTATCTT | |
| fwd-FANCF | ACACTCTTTCCCTACACGACGCTCTTCCGATC | 117 |
| | TAGCATTGCAGAGAGGCGTAT | |
| rev-FANCE | GTGACTGGAGTTCAGACGTGTGCTCTTCCGAT | 118 |
| | CTATGGATGTGGCGCAGGTAG | |
| fwd-HBG | ACACTCTTTCCCTACACGACGCTCTTCCGATC | 119 |
| | TCCTTTTATTCTTCATCCCTAGCC | |
| rev-HBG | GTGACTGGAGTTCAGACGTGTGCTCTTCCGAT | 120 |
| | CTGGAATGACTGAATCGGAACAA | |
| fwd-HEK293_site 2 | ACACTCTTTCCCTACACGACGCTCTTCCGATC | 121 |
| | TGGACGTCTGCCCAATATGTAA | |
| rev-HEK293_site 2 | GTGACTGGAGTTCAGACGTGTGCTCTTCCGAT | 122 |
| | CTAAGGGGGAAAAATTGTCCAG | |
| fwd-HEK293_site 3 | ACACTCTTTCCCTACACGACGCTCTTCCGATC | 123 |
| | TAAACGCCCATGCAATTAGTC | |
| rev-HEK293_site 3 | GTGACTGGAGTTCAGACGTGTGCTCTTCCGAT | 124 |
| | CTCCAGCCAAACTTGTCAACC | |
| fwd-HEK293_site 4 | ACACTCTTTCCCTACACGACGCTCTTCCGATC | 125 |
| | TCTCCCTTCAAGATGGCTGAC | |
| rev-HEK293_site 4 | GTGACTGGAGTTCAGACGTGTGCTCTTCCGAT | 126 |
| | CTAACGGAGACACACACACAGG | |
| fwd-HPRT_Exon 4 | ACACTCTTTCCCTACACGACGCTCTTCCGATC | 127 |
| | TTTGAAGTTTGTGTGTGTACATAAGGA | |
| rev-HPRT_Exon 4 | GTGACTGGAGTTCAGACGTGTGCTCTTCCGAT | 128 |
| | CTTGATTGATTGAAAGCACACTGTT | |
| fwd-HPRT Exon 8 | ACACTCTTTCCCTACACGACGCTCTTCCGATC | 129 |
| | TGAGAGGCACATTTGCCAGTAT | |
| rev-HPRT Exon 8 | GTGACTGGAGTTCAGACGTGTGCTCTTCCGAT | 130 |
| | CTTATGAGGTGCTGGAAGGAGAA | |
| fwd-VEGFA | ACACTCTTTCCCTACACGACGCTCTTCCGATC | 131 |
| | TACAGGGAAGCTGGGTGAAT | |
| rev-VEGFA | GTGACTGGAGTTCAGACGTGTGCTCTTCCGAT | 132 |
| | CTCACACGCACACACTCACTCA | |
| fwd- | ACACTCTTTCCCTACACGACGCTCTTCCGATC | 133 |
| PPP1R12C_site 3 | TGGAATTCTGCATCATGTGGGCGTGTCC | |
| rev- | GTGACTGGAGTTCAGACGTGTGCTCTTCCGAT | 134 |
| PPP1R12C_site 3 | CTGGATCAGCCAGGAACGAAGACCACTGG | |
| fwd- | ACACTCTTTCCCTACACGACGCTCTTCCGATC | 135 |
| PPP1R12C_site 4 | TAAGACAATCCTAGGAAGCAGGGTCAGC | |
| rev- | GTGACTGGAGTTCAGACGTGTGCTCTTCCGAT | 136 |
| PPP1R12C_site 4 | CTGGGCCAACATCGCCGCCG | |
| fwd- | ACACTCTTTCCCTACACGACGCTCTTCCGATC | 137 |
| PPP1R12C_site 6 | TGCCAGGCAGATAGACCAGACTGAGC | |
| rev- | GTGACTGGAGTTCAGACGTGTGCTCTTCCGAT | 138 |
| PPP1R12C_site 6 | CTGCCTCCTTACCATTCCCCTTCGACC | |
| fwd-EMX1_GGT PAM | ACACTCTTTCCCTACACGACGCTCTTCCGATC | 139 |
| | TTTCTTCCCACCTGGAATGTC | |
| rev-EMX1_GGT PAM | GTGACTGGAGTTCAGACGTGTGCTCTTCCGAT | 140 |
| | CTGCACTGACTGGACACAAGTGA | |
| fwd-VEGFA_CGT PAM | ACACTCTTTCCCTACACGACGCTCTTCCGATC | 141 |
| | TGGTCACTCCAGGATTCCAATAG | |
| rev-VEGFA_CGT PAM | GTGACTGGAGTTCAGACGTGTGCTCTTCCGAT | 142 |
| | CTCCAAGGTTCACAGCCTGAAA | |
| fwd-GRIN2B_AGA PAM | ACACTCTTTCCCTACACGACGCTCTTCCGATC | 143 |
| | TCCTCCTTTGTCTCTGCCTGT | |
| rev-GRIN2B_AGA PAM | GTGACTGGAGTTCAGACGTGTGCTCTTCCGAT | 144 |
| | CTGGATCTACATCACGTAACCTGTCTT | |
| fwd-FANCF_GGA PAM | ACACTCTTTCCCTACACGACGCTCTTCCGATC | 145 |
| | TCCGATGAGGAGACACTCCAA | |
| rev-FANCF_GGA PAM | GTGACTGGAGTTCAGACGTGTGCTCTTCCGAT | 146 |
| | CTCCACAGGCTGCTGAGAAAC | |
| fwd-PTEN_GGA PAM | ACACTCTTTCCCTACACGACGCTCTTCCGATC | 147 |
| | TAGATTTCTAAGCCACAGAAAAAGA | |
| rev-PTEN_GGA PAM | GTGACTGGAGTTCAGACGTGTGCTCTTCCGAT | 148 |
| | CTTGGTGACCAGCATTTTATGG | |
| fwd-MECP2_AGT PAM | ACACTCTTTCCCTACACGACGCTCTTCCGATC | 149 |
| | TAATGGAAGATCCAGAGAAAGC | |
| rev-MECP2_AGT PAM | GTGACTGGAGTTCAGACGTGTGCTCTTCCGAT | 150 |
| | CTTGCTACTCCAGTGAACACACAA | |

Table 7 below shows the primers used for analysis of DNA or RNA off-target editing. The 5′ tail sequences for extension of the HT True seq index are underlined.

TABLE 7

| Primer | Sequences | SEQ NO. |
|---|---|---|
| fwd-HBG OT1 | ACACTCTTTCCCTACACGACGCTCTTCCGATCTGCT | 151 |
| | GGGGAGAGAAAAGGAAA | |
| rev-HBG OT1 | GTGACTGGAGTTCAGACGTGTGCTCTTCCGATCTGA | 152 |
| | CTGTGCAAATGCTTCTCG | |
| fwd-HBG OT2 | ACACTCTTTCCCTACACGACGCTCTTCCGATCTAGG | 153 |
| | CTATTGGTCAAGGCAAG | |
| rev-HBG OT2 | GTGACTGGAGTTCAGACGTGTGCTCTTCCGATCTAC | 154 |
| | GGCTGACAAAAGAAGTCC | |
| fwd-HBG OT3 | ACACTCTTTCCCTACACGACGCTCTTCCGATCTGCT | 155 |
| | GGGGAGAGAAAAGGAAA | |
| rev-HBG OT3 | GTGACTGGAGTTCAGACGTGTGCTCTTCCGATCTGA | 156 |
| | CTGTGCAAATGCTTCTCG | |
| fwd-HBG OT4 | ACACTCTTTCCCTACACGACGCTCTTCCGATCTGCC | 157 |
| | AGCTAACATGGGTCATT | |
| rev-HBG OT4 | GTGACTGGAGTTCAGACGTGTGCTCTTCCGATCTGC | 158 |
| | CAGAATCGAGCACTGAG | |
| fwd-HBG OT5 | ACACTCTTTCCCTACACGACGCTCTTCCGATCTCCT | 159 |
| | TGGCTTGCTATTTGGAG | |
| rev-HBG OT5 | GTGACTGGAGTTCAGACGTGTGCTCTTCCGATCTGG | 160 |
| | TTCCCTCAAAACCTCCTC | |
| fwd-HBG OT6 | ACACTCTTTCCCTACACGACGCTCTTCCGATCTTAG | 161 |
| | CCACTGTCCTGGCTTCT | |
| rev-HBG OT6 | GTGACTGGAGTTCAGACGTGTGCTCTTCCGATCTGC | 162 |
| | TTCCCAGTGACACACAGA | |
| fwd-VEGFA3 | ACACTCTTTCCCTACACGACGCTCTTCCGATCTGCT | 163 |
| OT1 | CTCCACCTCGATGTCA | |
| rev-VEGFA3 | GTGACTGGAGTTCAGACGTGTGCTCTTCCGATCTAT | 164 |
| OT1 | CCGGTGCTGCAGTGAG | |
| fwd-VEGFA3 | ACACTCTTTCCCTACACGACGCTCTTCCGATCTTTG | 165 |
| OT2 | CTGTGTCTTCCTTCTGC | |
| rev-VEGFA3 | GTGACTGGAGTTCAGACGTGTGCTCTTCCGATCTTT | 166 |
| OT2 | GGTGGGACTGATGATGAA | |
| fwd-VEGFA3 | ACACTCTTTCCCTACACGACGCTCTTCCGATCTCAG | 167 |
| OT3 | AAGTGGTTGATGAATTGC | |
| rev-VEGFA3 | GTGACTGGAGTTCAGACGTGTGCTCTTCCGATCTGA | 168 |
| OT3 | AACCATTTGGGCTTTCCT | |
| fwd-HPRT E8 | ACACTCTTTCCCTACACGACGCTCTTCCGATCTTTG | 169 |
| OT | CTGAGCTCTTATATGTGCAA | |
| rev-HPRT E8 | GTGACTGGAGTTCAGACGTGTGCTCTTCCGATCTTT | 170 |
| OT | CTCTTGCCCTGACCATTC | |
| fwd-HEK4 OT1 | ACACTCTTTCCCTACACGACGCTCTTCCGATCTAGA | 171 |
| | CTCATAGCTGGGGCTGA | |
| rev-HEK4 OT1 | GTGACTGGAGTTCAGACGTGTGCTCTTCCGATCTCC | 172 |
| | GGATGATTCTCCTACTTCC | |
| fwd-HEK4 OT2 | ACACTCTTTCCCTACACGACGCTCTTCCGATCTCAA | 173 |
| | GGTCTGAGGCTCGAATC | |
| rev-HEK4 OT2 | GTGACTGGAGTTCAGACGTGTGCTCTTCCGATCTAG | 174 |
| | AGCAAACCTTGGCATTGT | |
| fwd-HEK4 OT3 | ACACTCTTTCCCTACACGACGCTCTTCCGATCTTGA | 175 |
| | GCTCATTTCCACCAGAA | |
| rev-HEK4 OT3 | GTGACTGGAGTTCAGACGTGTGCTCTTCCGATCTAT | 176 |
| | GGGAAATACGGGCTTAGG | |
| fwd-AARS1 | ACACTCTTTCCCTACACGACGCTCTTCCGATCTTCA | 177 |
| | GTTTTGCCCGATCTCCT | |
| rev-AARS1 | GTGACTGGAGTTCAGACGTGTGCTCTTCCGATCTCC | 178 |
| | CATGGTGCCTGACTAACA | |
| fwd-RSL1D1 | ACACTCTTTCCCTACACGACGCTCTTCCGATCTTGG | 179 |
| | CTTTCCAAATCAGTGGGTC | |
| rev-RSL1D1 | GTGACTGGAGTTCAGACGTGTGCTCTTCCGATCTCT | 180 |
| | CATAAGCTTAGACCAACAAGC | |
| fwd-CIP1 | ACACTCTTTCCCTACACGACGCTCTTCCGATCTGCT | 181 |
| | GAGGGCCATCTGAAACA | |
| rev-CIP1 | GTGACTGGAGTTCAGACGTGTGCTCTTCCGATCTAG | 182 |
| | GGTGCCTTCATGGTTAGC | |
| fwd-PERP | ACACTCTTTCCCTACACGACGCTCTTCCGATCTCTG | 183 |
| | TGGTGGAAATGCTCCCAAG | |
| rev-PERP | GTGACTGGAGTTCAGACGTGTGCTCTTCCGATCTCA | 184 |
| | CTCTCAGGAAGACAAGCATC | |
| fwd-TOPORS | ACACTCTTTCCCTACACGACGCTCTTCCGATCTTCA | 185 |
| | GTTTTGCCCGATCTCCT | |
| rev-TOPORS | GTGACTGGAGTTCAGACGTGTGCTCTTCCGATCTCC | 186 |
| | CATGGTGCCTGACTAACA | |

### 10. Confirmation of Ribonucleoprotein (RNP) Delivery Through Immunofluorescence Analysis

Mice were sacrificed at indicated time points after subretinal injection, and RPE-choroid-scleral complex was prepared. The complex was then treated with anti-FLAG (cat no. MA1-142-A488, Thermo), anti-ZO-1 (cat no. 339194, Thermo), or anti-RPE65 (cat no. NB100-355AF488, Novus) antibody, and observed under a confocal microscope. Nuclei were identified using 4′,6-diamidino-2-phenylindole (cat. no. D9542, Sigma).

11. Confirmation of Gene Expression Levels Via qRT-PCR (Quantitative Real-Time Polymerase Chain Reaction)

Total RNA was prepared from RPE cells using TRIzol reagent (cat no. 15596018, Thermo) 5 weeks after subretinal injection of ABE RNP/Lipofectamine 2000 mixture. The quality and quantity of extracted RNA were evaluated with a NanoDrop 2000 spectrophotometer (Thermo). cDNA was prepared using the High-Capacity RNA-to-cDNA kit (cat no. 4387406, Thermo). qRT-PCR was performed by the StepOnePlus Real-Time PCR System (Thermo) using TaqMan Fast Advanced Master Mix (cat no. 4444556, Thermo) and Gene Espression Assay (Thermo). The product IDs of gene expression analysis are as follows: Mm00504133_m1 for Rpe65; Mm99999915_g1 for Gapdh; and Mm03928990_g1 for Rn18s. Relative Rpe65 gene expression levels were normalized to the gene expression levels of Gapdh and Rn18s. All procedures were performed according to the Minimum Information for Publication of Quantitative Real-Time PCR Experiments guidelines.

### Example 1: Preparation of ABE/CBE RNP Complex in Human Cell Expression System

### 1.1 Preparation of High Purity ABE/CBE Protein

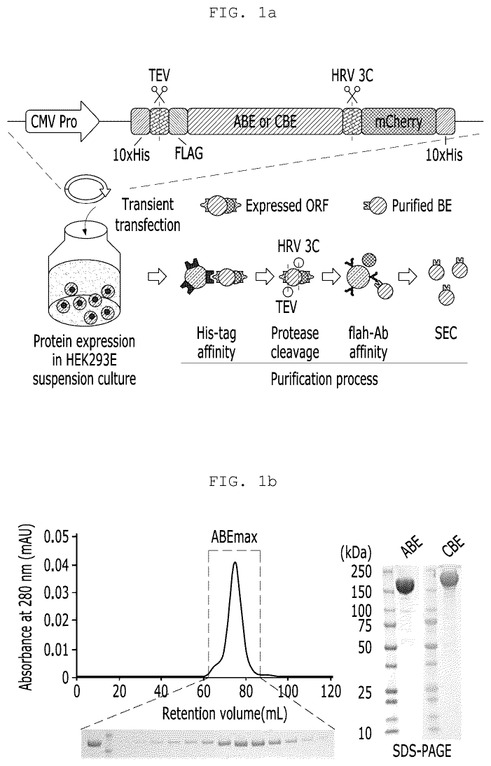

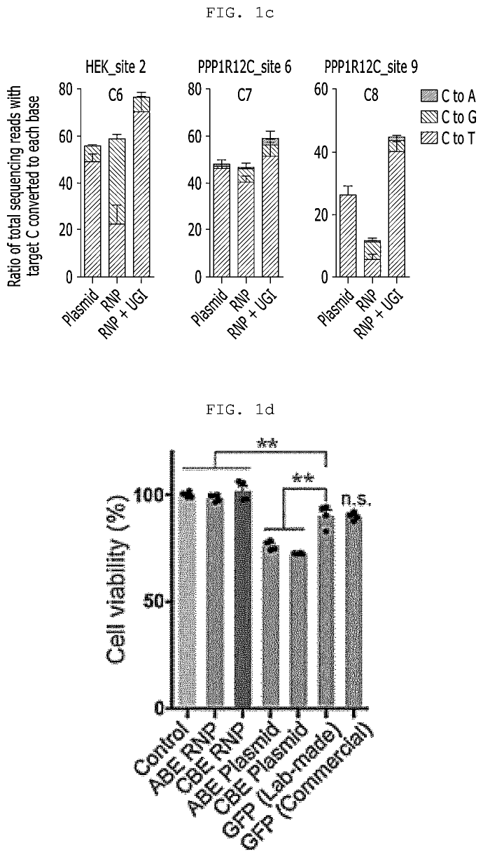

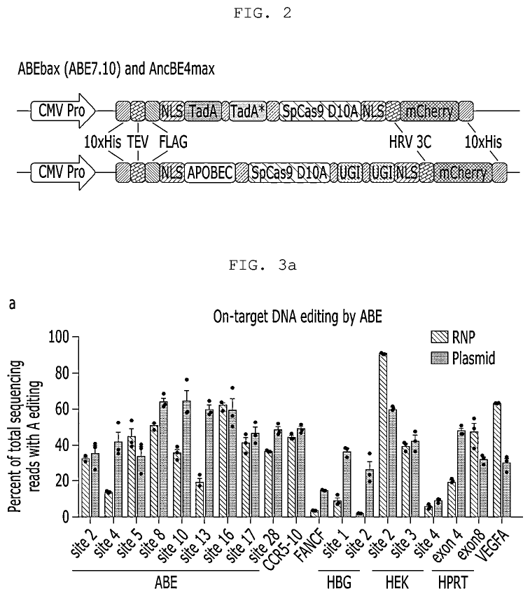

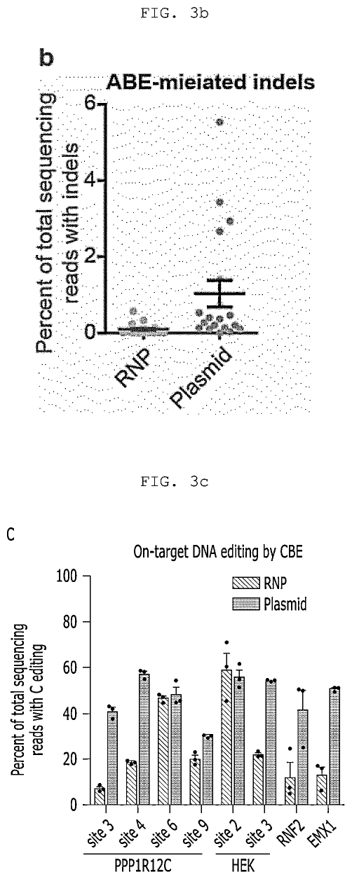

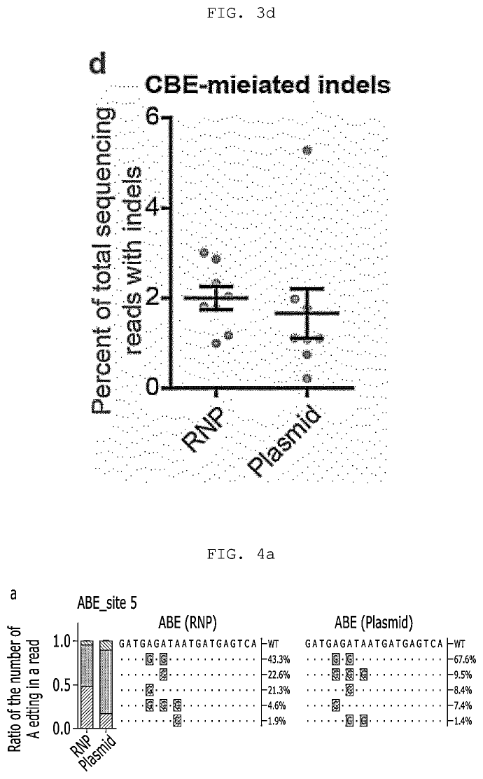

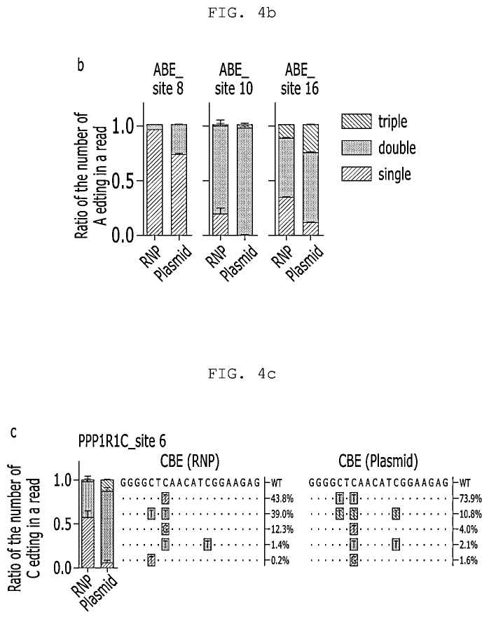

To prepare recombinant base editor proteins in human cell systems, sequences encoding optimized forms of ABE and CBE (i.e., ABEmax and AncBE4max), respectively, were cloned into the mammalian expression vector pEX-FlagR. This vector was designed for the fusion of red fluorescent protein (mCherry) and a dual affinity tag to the target protein, respectively, which facilitates convenient monitoring of protein expression and efficient protein purification (FIGS. 1a and 2).

The prepared plasmids were transfected into HEK293E cells in suspension culture, and ABE/CBE proteins were purified using Ni-affinity, anti-FLAG-M1-affinity, and size exclusion chromatography techniques sequentially. The polyhistidine (poly-His) and mCherry tags were cleaved by protease cleavage during purification, and thus purified ABE/CBE proteins with only the N-terminal FLAG tag remaining were obtained. By using this expression and purification method, 1 mg of highly pure CBE or ABE protein was reproducibly obtained from 1 liter of cell culture (FIG. 1b).

### 1.2 Confirmation of Activity of Purified ABE/CBE Proteins

To confirm the activity of purified ABE/CBE proteins, ABE RNPs targeting 19 different endogenous sites and CBE RNPs targeting 8 different endogenous sites were tested in HEK293T cells. In addition, as a control, ABE/CBE encoding plasmid was transfected.

According to high throughput sequencing (HTS) data obtained from bulk cell populations 3 days after transfection, ABE/CBE RNPs exhibited effective editing activity, indicating that ABE/CBE proteins were successfully produced in human cell systems.

CBE contained uracil DNA glycosylase inhibitor (UGI) to improve editing efficiency. Therefore, it could be appreciated that the concentration of UGI fused with CBE in RNP was relatively lower than that of the plasmid-encoded version, which was insufficient to inhibit uracil N-glycosylase activity in cells.

Further, when UGI was overexpressed, the overall CBE RNP editing activity was improved, and the ratio of products containing C to T editing (on-target) was increased compared to products containing any C editing (off-target) (FIG. 1c).

Further, ABE and CBE RNPs showed higher cell viability after transfection than ABE and CBE encoding plasmids (FIG. 1d). When transfecting with the GFP vector (˜3.5 kb) as a control, a slight decrease in cell viability was observed, indicating that the large size of the ABE/CBE plasmid (˜9 kb) could be a contributing factor to the decrease in cell viability. In other words, it can be considered that large-sized plasmids (6˜16 kbp) exhibit low viability and transfection efficiency.

These results indicate that the RNP system may be suitable for medical treatment.

### Example 2: Characterization of Base Editing

### 2.1 Analysis of Base Editing Active Window and Base Editing Pattern

An analysis was performed to determine whether the editing activity window and editing pattern derived from ABE/CBE RNP and ABE/CBE plasmid could be differentiated.

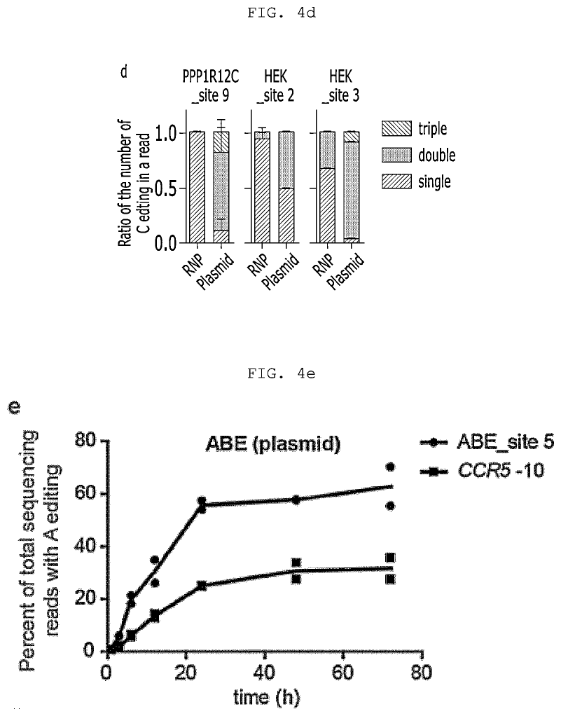

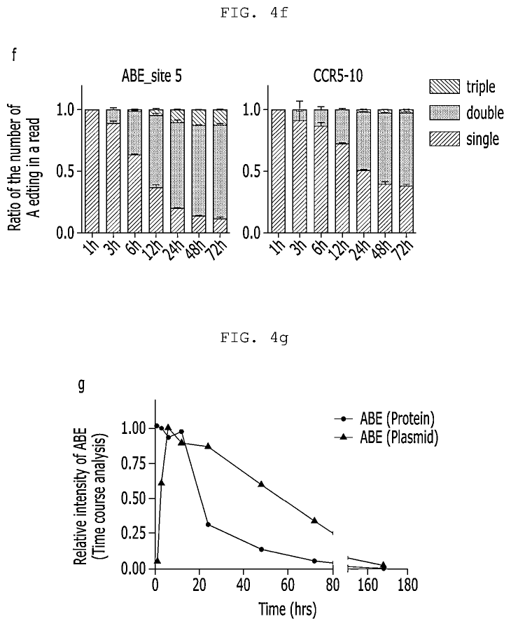

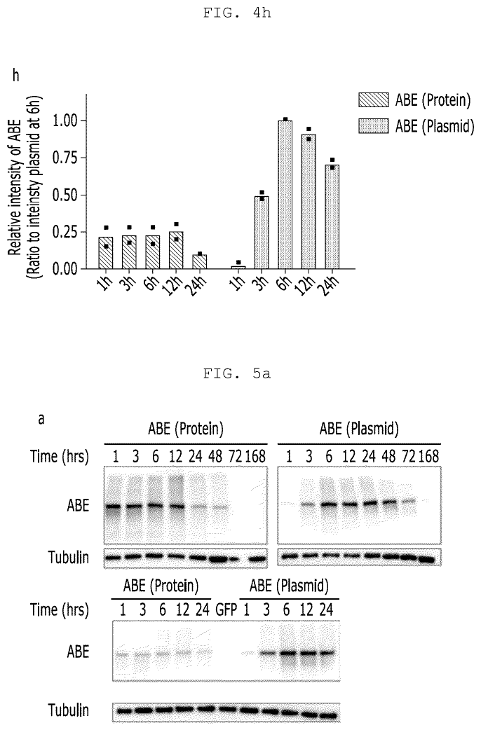

As a result of analyzing the base editing data of the RNP and plasmid-encoded base editors, the preferred editing activity windows of the RNP and plasmid-encoded base editors were similar (results not shown), but the editing patterns were different (FIGS. 4a to 4d). In general, RNP-encoded base editors converted fewer bases in the allele than plasmid-encoded base editors. In the case of ABE site 5 (A5) (FIG. 4a), single base conversions (‘ [symbol] ’ in the bars) were 47.8% of ABE RNP-treated cells but only 16.4% of plasmid-treated cells, and multiple base conversions (‘ [symbol] ’ and ‘ [symbol] ’ in the bars) occurred more frequently in the case of delivery through plasmid compared to delivery through RNP. This trend was also observed for most other targets (A8, A10, and A16;

FIG. 4b) and for CBE (FIGS. 4c and 4d).

### 2.2 Relationship Between Cell Lifespan and Base Editing

To investigate the reasons for the different base editing results, the frequency of different editing patterns derived from plasmid-encoded ABEs at different time points was measured (FIG. 4e). The ratio of multi-base conversions compared to single-base conversions gradually increased in a time-dependent manner (FIG. 4f), indicating that long-term expression of plasmid-encoded ABE affects multiple base conversions. In contrast, compared to plasmid-encoded ABE/CBE, ABE/CBE RNPs had fewer edits of bystander nucleotides, which is mainly because ABE/CBE RNPs have a shorter lifetime in cells.

### 2.3 Confirmation of Protein Expression Level

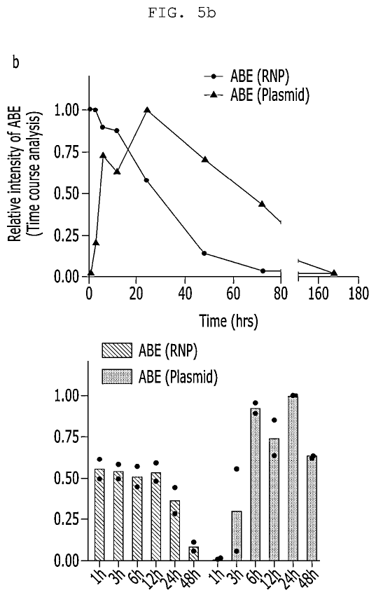

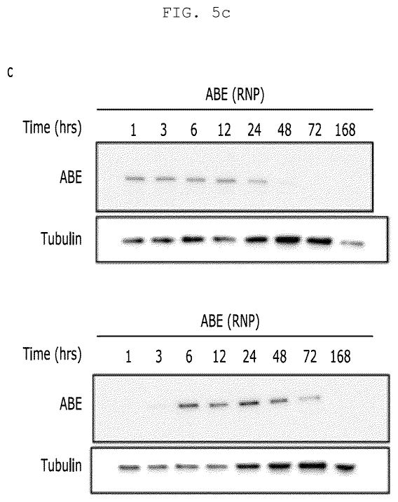

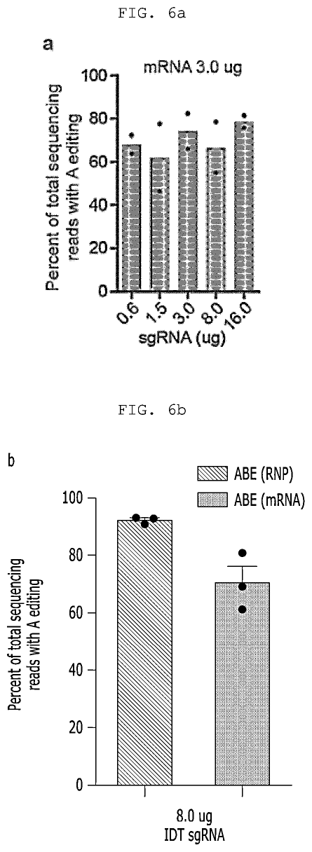

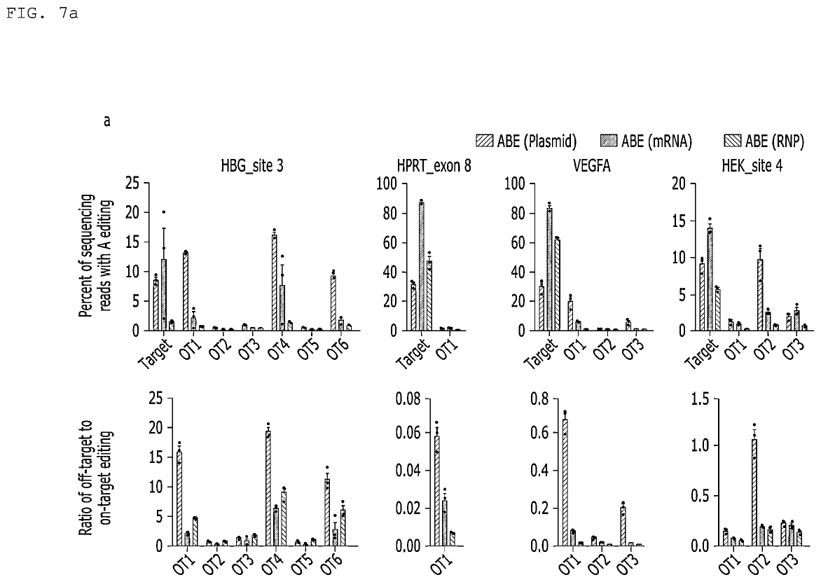

To determine the amount of ABE protein in cells from the two different delivery methods, ABE protein or ABE encoding plasmid were transfected without sgRNA, and ABE protein concentration in cell lysates was measured at different time points using Western blot analysis. In delivery experiments via protein, the initial ABE protein concentration was maintained for 12 hours and rapidly decreased at 24 hours. On the other hand, when delivered via plasmid, ABE concentration experienced a rapid increase within 6 hours, reaching its maximum level and maintaining that concentration until 24 hours. The concentration slowly decreased over the next 6 days (FIGS. 4g and 5a).

As a result of comparing the ABE protein abundance at each time point compared to the 6-hour time point in plasmid-transfected cells, the protein delivery-derived ABE protein concentration was always lower than the plasmid delivery-derived ABE concentration except for the first 3 hours (FIG. 4h).

In addition, ABE protein abundance was measured in the presence of sgRNA in cells after transfection of ABE RNP or ABE/sgRNA encoding plasmid (FIGS. 5b and 5c). The overall trend was similar to the above results without sgRNA, but it was found that ABE concentrations decreased more slowly for both the ABE RNP and ABE plasmid delivery methods. This is because the Cas9/sgRNA complex is more stable than the apo Cas9 protein in cells.

### Example 3: Confirmation of DNA Off-Target Effects

3.1 Confirmation of sgRNA-Dependent Off-Target DNA Editing Activity

First, the sgRNA-dependent off-target DNA editing activity, which is known to be triggered by the Cas9 effector portion of ABE, was evaluated.

In this experiment, an mRNA delivery method was used in addition to the RNP and plasmid delivery methods. mRNA was transcribed in vitro and sgRNA was synthesized at Integrated DNA Technologies (IDT), Inc. To optimize the concentration of ABE-encoding mRNA and sgRNA, several doses were tested for adenine base editing in HEK_site 2 to determine the most efficient conditions in HEK293T cells (i.e., 3.0 μg of each ABE-encoding mRNA and sgRNA) (FIG. 6).

Editing frequency was evaluated at both on- and off-targets after transfection of ABE RNP, ABE encoding mRNA, and ABE encoding plasmid for four defined targets (i.e., HBG_site 3, HPRT_exon 8, VEGFA, and HEK_site 4; Table 8).

Table 8 below shows the on-target and off-target sites used to analyze DNA-off-target editing activity. PAM (protospacer adjacent motif) is shown in italics. Mismatches or bulges to the on-target protospacer sequence are written in lowercase letters or hyphens. The most frequently edited A nucleotides in each target are underlined. In Table 8 below, “position” indicates a potential cleavage site (3 nt away from the PAM). In the Gene in Table 8 below, “OT” indicates Off-Target.

TABLE 8

| Gene | Seqeunces with PAM | Position | SEQ NO. |
|---|---|---|---|
| HBG_site 3 | GTGGGGAAGGGGCCCCCAAGAGG | chr11_5276202 | 187 |
| HBG_OT1 | GgtGGGAtGGGGtCCCCAAGTGG | chr3_13705838 | 188 |
| HBG_OT2 | GgtGGGgAGcGGCCCCCcAGTGG | chr9_138419302 | 189 |
| HBG_OT3 | agtGGGgAGGcGCCCtCAAGTGG | chr15_84049561 | 190 |
| HBG_OT4 | GTGGGG-AGtGGCCCCCAAGAGG | chr10_73282210 | 191 |
| HBG_OT5 | GTGGGG-AGcGGCCCCCcAGTGG | chr9_138419300 | 192 |
| HBG_OT6 | aTGaGGAAGcGaCCCCCAAGAGG | chr3_4746491 | 193 |
| VEGFA | GGTGAGTGAGTGTGTGCGTGTGG | chr9_110103695 | 194 |
| VEGFA_OT1 | aGTGAGTGAGTGTGTGtGTGGGG | chr14_65569159 | 195 |
| VEGFA_OT2 | tGTGAGTaAGTGTGTGtGTGTGG | chr14_62078773 | 196 |
| VEGFA_OT3 | GGTGAGTGtGTGTGTGCaTGTGG | chr2_177463426 | 197 |
| HPRT_exon 8 | GAAGTATTCATTATAGTCAAGGG | chrX_134498649 | 198 |
| HPRT-E8_OT | GAAGCATTCATTATAGTCAAAGG | chr11_93649491 | 199 |
| HEK_site 4 | GGCACTGCGGCTGGAGGTGGGGG | chr20_32761949 | 200 |
| HEK4_OT1 | tGCACTGCGGCcGGAGGaGGTGG | chr20_61435489 | 201 |
| HEK4_OT2 | GGCACgaCGGCTGGAGGTGGGGG | chr10_125006288 | 202 |
| HEK4_OT3 | GGCAtcaCGGCTGGAGGTGGAGG | chr10_75343344 | 203 |

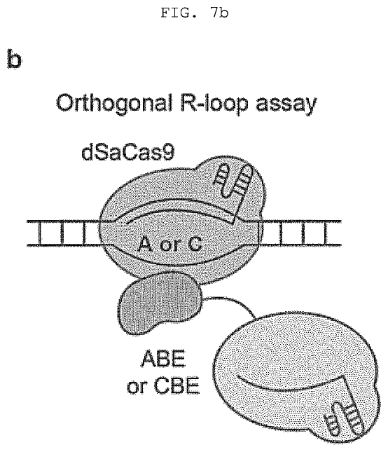

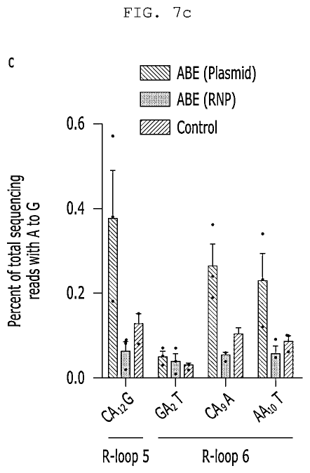

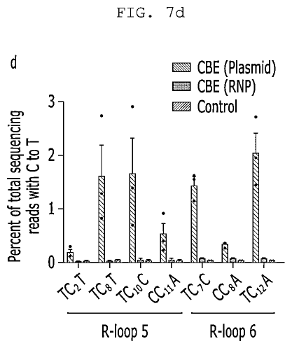

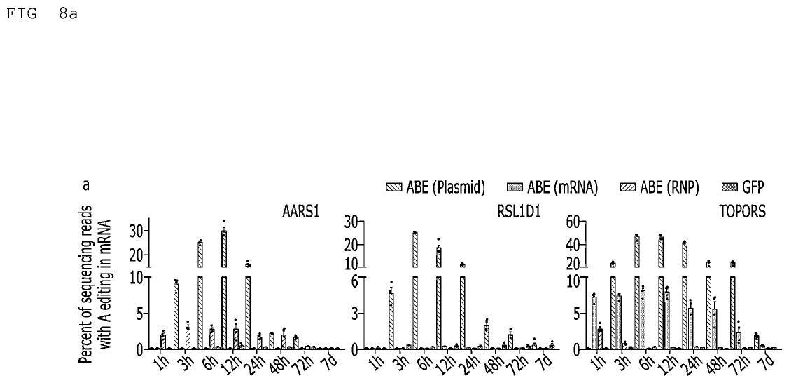

High-throughput sequencing (HTS) data results are shown in FIG. 7a. The plasmid delivery method had higher A-to-G proofreading efficiency than the RNP and mRNA delivery methods at HBG_site 3, but even in this case, the off-target rate was significantly higher than the RNP and mRNA delivery methods. Even when the RNP and mRNA delivery methods showed higher A-to-G correction efficiency than the plasmid delivery method in HPRT_exon 8, VEGFA, and HEK_site 4, the off-target (OT) rate was significantly reduced (FIG. 7a).

3.2 Confirmation of sgRNA-Independent Off-Target DNA Editing Activity

Next, sgRNA-independent promiscuous DNA deamination activity was confirmed for ABE or CBE. To accomplish this, an orthogonal R-loop assay was performed to form SaCas9 (dSaCas9) that is catalytically inactive in a single-stranded DNA region (FIG. 7b).

Table 9 below shows primers used in orthogonal R-loop analysis. The 20-nt target protospacers are underlined. The 5′ tail sequences for extension of the TruSeq HT index are shown in italics.

TABLE 9

| | | SEQ |
|---|---|---|
| Primer | Sequences | NO. |
| F-R-loop5 | 5′-CACCGTCTGCTTCTCCAGCCCTGGC-3′ | 204 |
| R-R-loop5 | 5′-AAATGCCAGGGCTGGAGAAGCAGAC-3′ | 205 |
| F-R-loop6 | 5′-CACCGGATGTTCCAATCAGTACGCA-3′ | 206 |
| R-R-loop6 | 5′-AAATTGCGTACTGATTGGAACATCC-3′ | 207 |
| fwd-R-loop | 5′-ACACTCTTTCCCTACACGACGCTCTTCC | 208 |
| site 5 | GATCTGCCTAGAAAGGCATGGATGA-3′ | |
| rev-R-loop | 5′-GTGACTGGAGTTCAGACGTGTGCTCTTC | 209 |
| site 5 | CGATCTAGCCCCTGTCTAGGAAAAGC-3′ | |
| fwd-R-loop | 5′-ACACTCTTTCCCTACACGACGCTCTTCC | 210 |
| site 6 | GATCTAAGTTGCCCAGAGTCAAGGA-3′ | |
| rev-R-loop | 5′-GTGACTGGAGTTCAGACGTGTGCTCTTC | 211 |
| site 6 | CGATCTCCCAGGTGCTGACGTAGGTA-3′ | |

From the results of orthogonal R-loop analysis, very little A editing was found in R-loop regions 5 and 6 in ABE RNP-transfected cells, whereas in ABE encoding plasmid-transfected cells, quite significant A editing was observed in the CA₁₂G, CA₉A, and AA₁₀T motifs of two R-loop regions (sites 5 and 6) (FIG. 7c). Likewise, a similar trend was observed in cells transfected with CBE RNP and CBE encoding plasmid (FIG. 7d).

These results indicate that delivery via RNP leads to much more precise and specific base editing.

### Example 4: Confirmation of RNA Off-Target Effects in Transcriptome

In Example 4, promiscuous ABE-mediated RNA deamination activity was confirmed according to each delivery method.

To accomplish this, adenine mutation frequencies were calculated in complementary DNA (cDNA) derived from RNA transcripts (AARS1, RSL1D1, and TOPORS; Table 10) after transfection of ABE RNP, ABE-encoding mRNA, or ABE-encoding plasmid in the presence of sgRNA targeting HEK_site 2.

Table 10 below shows the RNA off-target sites used in Example 4. Edited A nucleotides are underlined. In Table 10 below, “position” indicates the position of each edited A nucleotide.

TABLE 10

| Gene | Sequences | Position | SEQ NO. |
|---|---|---|---|
| AARS1 | CCACTACGACCGGATTGGTG | chr16_70271877 | 212 |
| RSL1D1 | CGGCTACGGAATTTAGGAGA | chr16_11836797 | 213 |
| TOPORS | TTCATACGAAGAAGGCAGCC | chr9_32543383 | 214 |

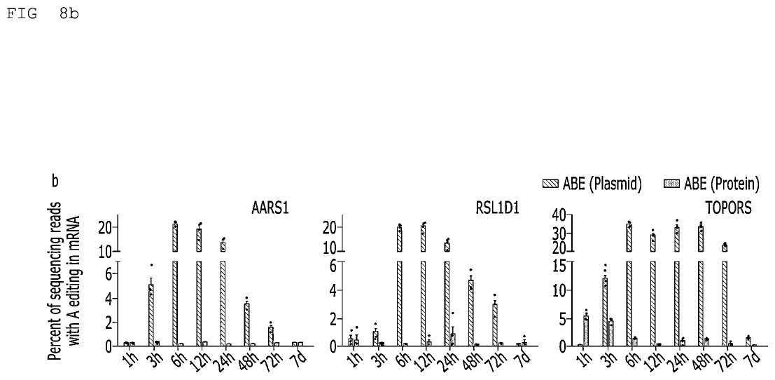

Results thereof are shown in FIG. 8. In cells transfected with RNP, ABE showed little RNA editing activity at the AARS1 and RSL1D1 sites at any time and minimal RNA editing activity at the TOPORS site for the first 3 hours, whereas cells transfected with mRNA or plasmid showed persistent promiscuous RNA deamination at all three sites for 3 days or more (FIG. 8a). To confirm the relationship between this RNA deamination activity and sgRNA, the same experiment was repeated without sgRNA, and it was confirmed that off-target RNA editing occurred similarly (FIG. 8b).

From this, it was confirmed that the RNP of the present disclosure exhibits a very low off-target effect.

### Example 5: Verification of In Vivo Base Editing and Mutation Correction Effects

To demonstrate the in vivo use of ABE RNPs, in vivo DNA editing via ABE RNP delivery was confirmed.

### 5.1 Confirmation of Target Base Editing in Normal Mouse Through ABE RNP Delivery

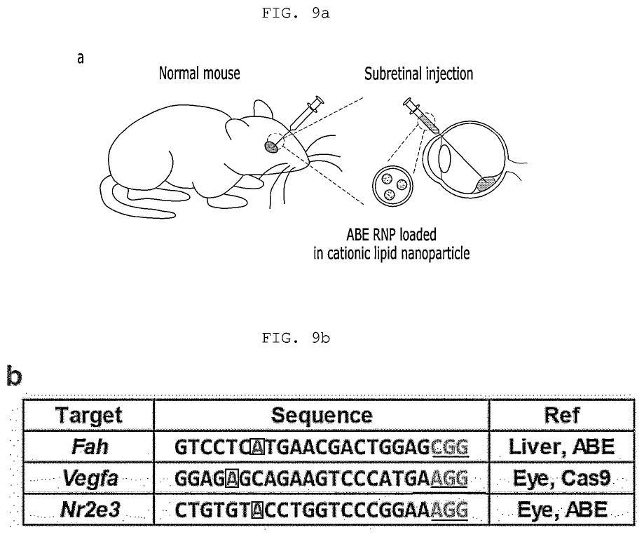

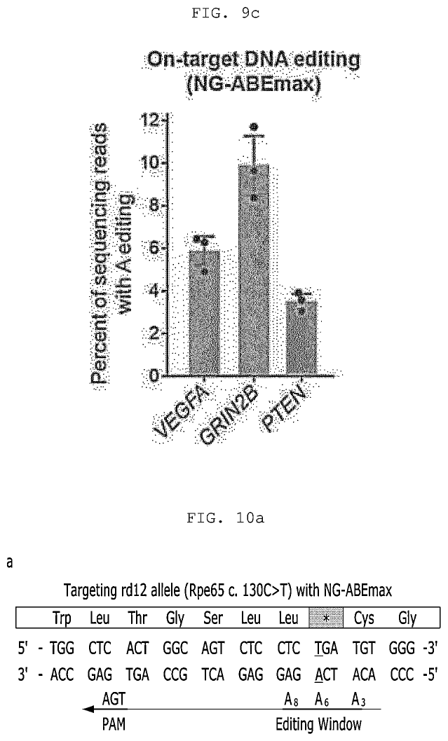

In the present experiment, the Fah, Vegfa, and Nr2e3 genes were targeted in normal mice. Purified ABEmax and sgRNA (synthesis of IDT) were injected into one eye of an adult mouse via subretinal injection with Lipofectamine 2000 (FIG. 9a). One week after injection, genomic DNA was isolated from retinal pigment epithelial cells (RPE cells) of RNP-injected mice, and high-throughput sequencing (HTS) data was obtained. The results showed clear base editing in Fah, Vegfa, and Nr2e3 (FIG. 9b).

### 5.2 Correction of Retinal Degeneration Caused by Rpe65 Gene Mutation

An attempt was made to correct a nonsense mutation in the Rpe65 gene causing retinal degeneration in rd12 mice.

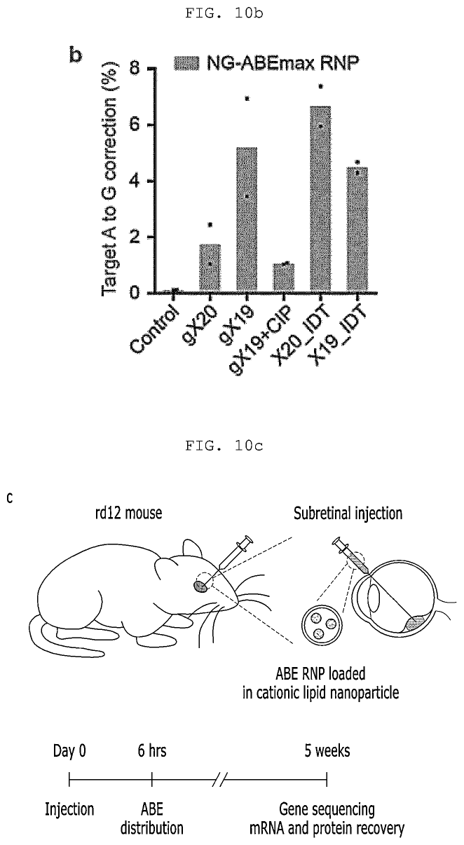

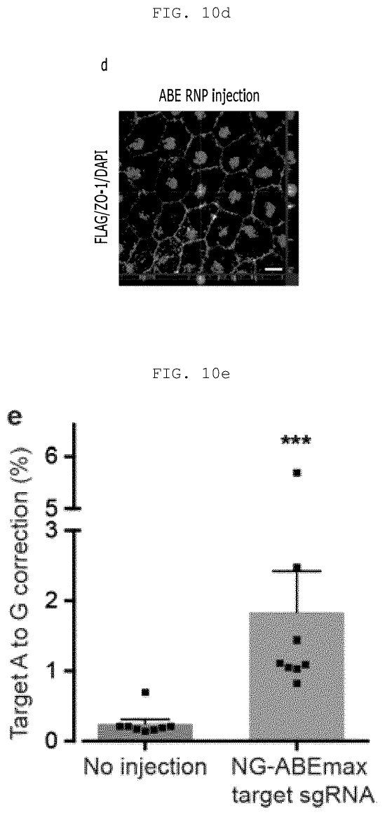

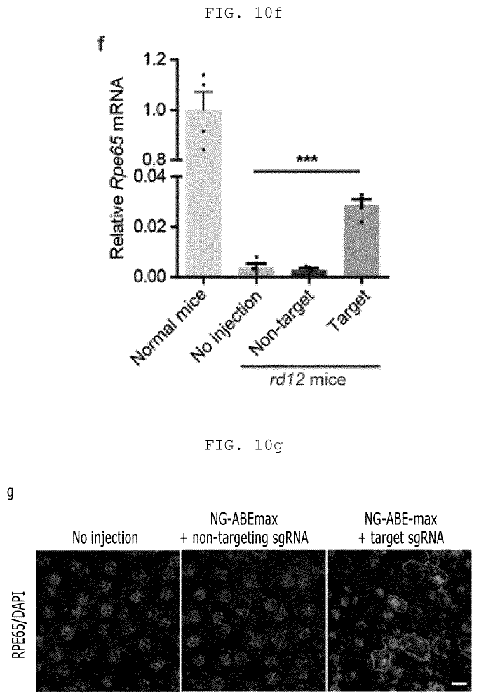

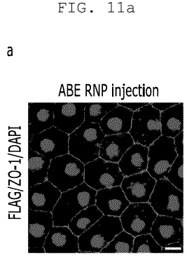

Since there was no relevant NGG protospacer adjacent motif (PAM) sequence near the mutation (FIG. 10a), seven additional mutations were inserted to construct the NG PAM targetable ABEmax (hereinafter referred to as NG-ABEmax) protein. After delivery in the form of NG-ABEmax RNP, a test was performed to determine whether the NG-ABEmax protein exhibits editing activity at three endogenous targets (VEGFA, GRIN2B, and PTEN). According to high-throughput sequencing (HTS) data, NG-ABEmax RNP was confirmed to exhibit editing activity on all three targets (FIG. 9c).

Then, sgRNA containing the TGA PAM and matching the disease-related Rpe65 point mutation (c.130C>T) to adenine (A6) at position 6 was designed (FIG. 10a). The efficiency when mutations were corrected was confirmed using NG-ABEmax RNPs with various types of sgRNA in rd12 mouse embryonic fibroblasts (rd12 mEF) (FIG. 10b). Even though there were differences in efficiency depending on the type of sgRNA used, it was confirmed that all of sgRNAs showed a correction effect. This indicates that the RNP of the present disclosure is usable with any known sgRNA.

As a result of FIG. 10b, IDT's chemically synthesized sgRNA (X20_IDT) showed the most effective editing activity, which was used for in vivo delivery to mice.

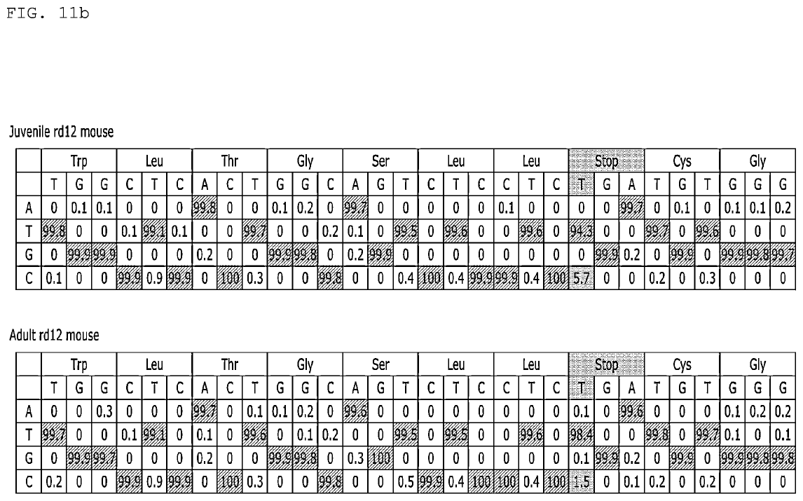

Ultimately, purified NG-ABEmax and X20 IDT sgRNA (hereinafter referred to as target sgRNA) were injected into one eye of an adult or juvenile rd12 mouse via subretinal injection along with Lipofectamine 2000 (FIG. 10c). It was found that while the injected NG-ABEmax RNPs were successfully localized in the cytoplasm and nucleus in the RPE at 6 hours after injection (FIG. 10d), NG-ABEmax RNPs were not detected in the RPE at 72 hours after injection (FIG. 11a), indicating that RNPs were degraded as rapidly in cells in vivo as in cultured cells (FIG. 4g).

In particular, according to high-throughput sequencing (HTS) data of genomic DNA isolated from the RPE of NG-ABEmax RNP-injected mice, the RNP of the present disclosure showed no bystander editing and precise correction of the rd12 mutation (FIG. 11b).

Restoration of a functional Rpe65 gene was verified at both mRNA and protein levels. RPE of rd12 mice treated with ABE RNP containing targeting sgRNA showed a significant increase in Rpe65 mRNA expression. The expression level in these mice was 2.8% of that in normal mice, which was significantly higher than that in untreated rd12 mice (no injection) or ABE RNP (non-target) treated rd12 mice containing non-targeting sgRNA (FIG. 10f). Further, it was confirmed that Rpe65 was expressed in the RPE of NG-ABEmax RNP-treated rd12 mice (FIG. 10g), indicating successful gene correction of mutant Rpe65.

1. A method for preventing or treating retinal dysfunction comprising administering to a subject in need thereof
(i) a fusion protein comprising Cas9 domain; and an adenine deaminase domain or a cytidine deaminase domain; and
(ii) a guide RNA (gRNA).

2. The method of claim 1, wherein the fusion protein further comprises a uracil glycosylase inhibitor (UGI) domain.

3. The method of claim 1, wherein the Cas9 domain is a Cas9 nickase (nCas9) that cleaves a nucleotide target strand in a nucleotide duplex.

4. The method of claim 1, wherein the fusion protein is an adenine base editor (ABE) combining adenine deaminase or a cytosine base editor (CBE) combining cytosine deaminase.

5. The method of claim 1, wherein the Cas9 domain recognizes a protospacer adjacent motif (PAM) of a target nucleic acid sequence, and the PAM is NGG or NG.

6. The method of claim 1, wherein the gRNA is a single guide RNA (sgRNA).

7. The method of claim 1, wherein the fusion protein and the gRNA are present in the form of a ribonucleoprotein (RNP) complex, and the gRNA is bound to the Cas9 domain.

8. The method of claim 1, wherein the retinal dysfunction is caused by a point mutation in a gene.

9. The method of claim 8, wherein the fusion protein or complex corrects the point mutation.

10. The method of claim 9, wherein the point mutation comprises a T to C point mutation and deamination of the mutant C base results in a sequence not associated with the retinal dysfunction; or
the point mutation comprises a G to A point mutation, and deamination of the mutant A base results in a sequence not associated with the retinal dysfunction.

11. The method of claim 1, wherein the gRNA comprises a sequence of contiguous nucleotides complementary to a target sequence associated with retinal dysfunction.

12. The method of claim 1, wherein the gRNA targets a gene associated with retinal dysfunction.

13. The method of claim 1, wherein the retinal dysfunction is a retinal degenerative disease; retinitis pigmentosa; retinitis pigmentosa; angiopathy; Drusen; lebers congenital amaurosis; hereditary or acquired macular degeneration; age-related macular degeneration (AMD); Best disease; retinal detachment; cerebral atrophy; choroidal defect; pattern dystrophy; retinal pigment epithelium (RPE) dystrophy; Stargardt disease; retinal pigment epithelium (RPE) and retinal damage caused by any of light, laser, infection, radiation, neovascularization or traumatic injury; retinal dysplasia; color blindness; choroideremia; myopic choroidal neovascularization; nodular choroidal angiopathy; central serous chorioretinopathy; macular hole; macular dystrophy; diabetic retinopathy; retinal arteriovenous occlusion; hypertensive retinopathy; retinal aortic aneurysm; ophthalmic ischemia syndrome; retinopathy of prematurity; acute retinal necrosis; cytomegalovirus retinitis; toxoplasma retinochoroiditis; syphilitic chorioretinitis; retinal detachment; or retinoblastoma.

14. A purification method of a fusion protein, comprising:
(a) expressing a fusion protein in a cell, the fusion protein containing a Cas9 domain; and an adenine deaminase domain or a cytidine deaminase domain, wherein the fusion protein further includes an affinity tag;
(b) dissolving the fusion protein expressed in Step (a) to produce a lysate;
(c) performing primary purification on the lysate of Step (b) by affinity chromatography;
(d) performing secondary purification on the fusion protein purified in Step (c) by affinity chromatography; and
(e) performing third purification on the fusion protein purified in Step (d) by size exclusion chromatography.

15. The purification method of claim 14, wherein the affinity tag is a polyhistidine tag or a FLAG tag.

16. The purification method of claim 14, wherein the fusion protein of Step (a) comprises a 10×His-Flag tag at the N-terminus and an mCherry-10×His tag at the C-terminus.

17. The purification method of claim 14, wherein Step (c) comprises contacting the lysate of Step (b) with a Ni-NTA resin, wherein the fusion protein is bound to the Ni-NTA resin.

18. The purification method of claim 14, wherein Step (d) comprises contacting the fusion protein purified in Step (c) with an α-FLAG M1 agarose resin, wherein the fusion protein is bound to the α-FLAG M1 agarose resin.

19. The purification method of claim 14, wherein the size exclusion chromatography of Step (e) is performed on a HiLoad 16/600 Superdex 200 pg, HiLoad 26/600 Superdex 200 pg, or HiLoad 16/600 Superdex 75 pg column.

20. (canceled)

21. A ribonucleoprotein (RNP) complex comprising:
a fusion protein purified according to the purification method of claim 14; and
a guide RNA (gRNA) bound to a Cas9 domain of the fusion protein.

22. (canceled)
